(19) United States
(12) Patent Application Publication
(10) Pub. No.: US 2025/0066287 A1
(43) Pub. Date: Feb. 27, 2025

(54) PHASE CHANGE AROMATIC AZO COMPOUNDS, METHOD OF MANUFACTURE, AND USE AS HOMOGENOUS CATALYST

(71) Applicant: Brandeis University, Waltham, MA (US)

(72) Inventors: Grace G.D. Han, Belmont, MA (US); Qianfeng Qiu, Waltham, MA (US)

(21) Appl. No.: 18/732,172

(22) Filed: Jun. 3, 2024

Related U.S. Application Data
(60) Provisional application No. 63/505,602, filed on Jun. 1, 2023.

(51) Int. Cl. C07C 245/08; B01J 31/02; B01J 35/39; C07F 1/08; C07F 15/00

(52) U.S. Cl. CPC C07C 245/08; B01J 31/0237; B01J 35/39; C07F 1/08; C07F 15/0066

(57) ABSTRACT

A compound of Formula (I)

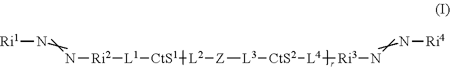

wherein in Formula (I), Ri¹, Ri², Ri³, and Ri⁴ are each independently an aryl or heteroaryl 5- or 6-membered ring optionally substituted with a C₁₋₆ alkyl, C₁₋₆ alkoxy, C₁₋₆ alkylthio, halogen, cyano, halogenated C₁₋₆ alkyl, halogenated C₁₋₆ alkoxy, or di(C₁₋₆ alkyl)amino; each L¹, L², L³, and L⁴ is independently a bond or a C₁₋₃₀ linking group optionally including a heteroatom; CtS¹ and CtS² are each independently catalytic moieties; Z is a moiety that increases crystallinity of the compound; and r is 0 to 4, wherein a first geometric isomer of the compound of Formula (I) has a first solubility in the organic solvent, and a second geometric isomer of Formula (I) has a second, different solubility in the organic solvent. Use of the compound of Formula (I) as a homogenous organocatalyst, or as a photoswitchable, homogenous organocatalyst, or as a recyclable, photoswitchable, homogenous organocatalyst.

### CROSS-REFERENCE TO RELATED APPLICATION

This application claims the benefit of U.S. Provisional Application No. 63/505,603, filed Jun. 1, 2023, which is incorporated herein by reference in its entirety.

### STATEMENT OF GOVERNMENT SUPPORT

This invention was made with government support under FA9550-22-1-0254 awarded by the Air Force Research Laboratory, and 2142887, and 1726346 awarded by the National Science Foundation. The government has certain rights in the invention.

### BACKGROUND

Disclosed herein are phase change aromatic azo compounds, methods of their manufacture, and uses thereof, particularly as homogenous catalysts that can be recycled.

Homogeneous catalysts display highly-desired properties such as high chemo-, regio-, and enantio-selectivity, yet the recovery and recycling of such catalysts have remaining challenges. The most commonly-used separation methods, e.g., distillation, chromatography, and extraction, while simple and fast, can result in the decomposition or loss of catalysts. The heterogenization of homogeneous catalysts, i.e., placing homogeneous catalysts on a solid support, can suffer from catalyst leaching and the loss of activity. A more widely applicable and adaptable method of catalyst recovery uses biphasic solution systems including organic/water systems, organic/ionic liquid systems, and organic/fluorous systems. In biphasic systems, a homogeneous and monophasic reaction condition can be achieved at high temperatures above the consolute temperature. Upon completion of the reaction, the biphasic structure is restored at a lowered temperature, and the catalysts are separated from the products, as a result of their orthogonal solubilities.

Solid-liquid fluorous biphasic systems have also been used, which enable the reaction in a single organic solvent, eliminating the use of highly-fluorinated solvents, as described, for example, by C. Rocaboy, J. A. Gladysz, New J. Chem. 2003, 27, 39-49; M. Wende, J. A. Gladysz, J. Am. Chem. Soc. 2003, 125, 5861-5872; C. Gimbert, A. Vallribera, J. A. Gladysz, M. Jurisch, Tetrahedron Lett. 2010, 51, 4662-4665; and N. Lu, W. C. Chung, H. F. Chiang, Y. C. Fang, L. K. Liu, Tetrahedron 2016, 72, 8508-8515. In these systems, the solid thermomorphic fluorous catalysts undergo a phase transition in an organic solvent by solubilizing at elevated temperatures to catalyze reactions, then precipitating out at lowered temperatures to be filtered and recovered. The temperature-dependent biphasic systems, however, have challenges such as the limited scope of reactions, reagents, and solvents that can accommodate a large temperature change.

A number of unrecoverable homogeneous catalysts have been developed that use light-triggered geometrical change of key sites in catalyst structures, for example those reported in Z. S. Kean, et al., Angew. Chem. Int. Ed. 2014, 53, 14508-14511; R. Dorel, B L. Feringa, Chem. Commun. 2019, 55, 6477-6486; M. Szewczyk, et al., ACS Catal. 2018, 8, 2810-2814; S. P. Ihrig, et al., Chem. Commun. 2019, 55, 4290-4298; R. S. Stoll, S. Hecht, Angew. Chem. Int. Ed. 2010, 49, 5054-5075; V. Blanco, et al., Chem. Soc. Rev. 2015, 44, 5341-5370; and T. Arif, et al. Catal. Sci. Technol. 2018, 8, 710-715. However, there are only a few reports describing the optical control of a catalyst aggregation state. One such report describes photoswitch-functionalized Au nanoparticles, which were used to show the optically-controlled exposure of metal surfaces, despite the gradual loss of photoswitch groups under irradiation (see, Y. Wei, S. Han, J. Kim, S. Soh, B. A. Grzybowski, J. Am. Chem. Soc. 2010, 132, 11018-110). Other studies with this focus include a nucleophilic catalyst that exhibits a reversible aggregation in the optimized set of mixed nonpolar solvents reported (see, A. Nojiri, N. Kumagai, M. Shibasaki, Chem. Commun. 2013, 49, 4628-4630), and an azobenzene-coordinated Pd complex for the optically-controlled Suzuki coupling, which, however, undergoes considerable degradation of catalyst and occurrence of side reactions (see, A. Kunfi et al., G. London, RSC Adv. 2021, 11, 23419-23429). Although these studies investigated optically controlled catalysts, the recovery or recycling of the catalysts remains to be explored.

There accordingly remains a need in the art for new homogenous catalysts that can be readily recycled and recovered upon light triggering.

### BRIEF SUMMARY

Disclosed herein is a compound of Formula (I)

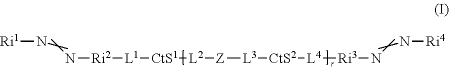

wherein in Formula (I),
Ri¹, Ri², Ri³, and Ri⁴ are each independently an aryl or heteroaryl 5- or 6-membered ring optionally substituted with a C₁₋₆ alkyl, C₁₋₆ alkoxy, C₁₋₆ alkylthio, halogen, cyano, halogenated C₁₋₆ alkyl, halogenated C₁₋₆ alkoxy, or di(C₁₋₆ alkyl)amino;
each L¹, L², L³, and L⁴ is independently a bond or a C₁₋₃₀ linking group optionally including a heteroatom;
CtS¹ and CtS² are each independently catalytic moieties;
Z is a moiety that increases crystallinity of the compound; and
r is an integer from 0 to 4; and
a first geometric isomer of the compound of Formula (I) has a first solubility in the organic solvent, and a second geometric isomer of Formula (I) has a second, different solubility in the organic solvent.

Also disclosed is use of the compound of Formula (I) (which includes a compound of Formula (II) and Formula (III)) as a homogenous organocatalyst, or as a photoswitchable, homogenous organocatalyst, or as a recyclable, photoswitchable, homogenous organocatalyst.

A method of catalyzing a reaction includes providing an organic reaction medium comprising the compound of Formulas (I), (II), or (III) having a first phase having a first solubility in an organic solvent, and a reactant subject to a catalytic transformation by the compound of Formulas (I), (II), or (III); irradiating the organic reaction medium with a first wavelength to effect a phase change of the compound of Formulas (I), (II), or (III) to a second phase having a second, higher solubility in the organic solvent; and catalyzing the transformation of the reactant to a product. At least two cycles of this method can be performed.

In another aspect, a method of catalyzing a reaction includes providing an organic reaction medium comprising the compound of Formulas (I), (II), or (III) in the form of a second phase having a higher solubility in an organic solvent than a first phase of the compound of Formulas (I), (II), or (III), and a reactant subject to a catalytic transformation by the compound of Formulas (I), (II), or (III); and catalyzing the transformation of the reactant to a product

In still another aspect, a method of catalyzing a reaction includes providing an organic reaction medium comprising the compound of Formulas (I), (II), or (III) in the form of a second phase having a higher solubility in an organic solvent than a first phase of the compound of Formulas (I), (II), or (III), and a reactant subject to a catalytic transformation by the compound of Formulas (I), (II), or (III); catalyzing the transformation of the reactant to a product; after the catalzying, irradiating the compound of Formulas (I), (II), or (III) in the form of the second phase with light of a second wavelength to effect a phase change of the compound of Formula (I) to the first phase having a lower solubility in the organic solvent; adding additional reactant to the compound of Formula (I) in the form of the first phase having a lower solubility in the organic solvent; and before or after adding the reactant, irradiating the compound of Formula (I) in the form of the first phase with light having a first wavelength to convert the compound of Formula (I) to the second phase having a higher solubility in an organic solvent. At least two cycles of this method can be performed.

The above described and other features are exemplified by the following figures, detailed description, examples, and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following Figures are exemplary embodiments, which are provided to illustrate the present disclosure. The Figures are not intended to limit devices made in accordance with the disclosure to the materials, conditions, or process parameters set forth herein.

FIG. 1 is a schematic representation of an exemplary photoswitchable biphasic catalyst system as described herein, that shows the reversible photo-isomerization of catalysts and their solubility changes, wherein R: reactant, P: product, hv1 and hv2: energy of photons at different frequencies (v1 and v2).

FIG. 2 shows DSC plots of E- and Z-isomers of compound 1.

FIG. 3 shows optical images of E- and Z- isomers of compound 1 at room temperature, showing the phase difference (crystalline solid E and liquid Z).

FIG. 4 shows a UV-vis absorption spectra of compound 1 in acetonitrile (about 1.7×, 10⁻⁵ M) before irradiation (100% E, black) and after irradiation at 340 nm (88% Z, red) and at 430 nm (82% E, blue).

FIG. 5 shows a UV-vis absorption spectra of compound 2 in methanol (about 1.6×10⁻⁵ M) before irradiation (100% E, black) and after irradiation at 340 nm (84% Z, red) and at 430 nm (84% E, blue).

FIG. 6 shows Z-to-E photoswitching kinetics (% Z in suspension, black) and catalyst recovery yields (red) of compounds 1 (solid circles) and 2 (hollow squares) upon 430 nm irradiation and consequent precipitation measured over irradiation time. After two hours, no further increase in recovery is observed.

FIG. 7A shows optical images showing the reversible solubility change between E and Z isomers of compound 1 in acetonitrile upon irradiation at 340 nm and 430 nm; and FIG. 7B shows optical images showing the reversible solubility change between E and Z isomers of compound 2 in methanol upon irradiation at 340 nm and 430 nm.

FIG. 8 is a schematic illustration of the reversible dissolution and precipitation of the photoswitchable catalyst in the presence of solvent, reagent, and product.

FIG. 9A is a scheme of an exemplary Michael addition reaction; FIG. 9B shows the product yields achieved with 10 mol % pre-activated soluble 1-Z, insoluble 1-E, and no catalyst in acetonitrile over 10 hours; and FIG. 9C shows the recovery rate of catalyst 1 (left, green) and product yield (right, blue) for three cycles of reactions. Reaction times for recycling testing in FIG. 9C were 16 hours for the first and second 2nd reaction and 24 hours for the third reaction to maximize yield of the product

FIG. 10A shows a scheme of an exemplary Henry reaction; FIG. 10B shows product yields achieved with 10 mol % pre-activated soluble 1-Z, in-situ UV activated 1, insoluble 1-E, and no catalyst in acetonitrile for 24 hours; and FIG. 10C shows the recovery rate of catalyst 1 (left, green) and percentage of product yield (right, blue) for three cycles of reactions. Reaction times for recycling testing in FIG. 10C were 16 hours for the first and second 2nd reaction and 24 hours for the third reaction to maximize yield of the product.

FIG. 11 shows multiCP/MAS solid-state ¹³C NMR spectra (thick black lines), and corresponding spectra after recoupled dipolar dephasing (thin red lines, scaled up by 1.11), wherein FIG. 11(a) shows a reference diacetylene diazobenzene, FIG. 11(b) compound 1, and FIG. 11(c) compound 2; the arrow marks a CH₂ of large-amplitude mobility. Bottom trace (green): Signal of carbons with fast ¹³C spin-lattice relaxation within a 2-s period. Stick spectra (orange) in (a) and (c): Chemical shifts predicted by quantum-chemical calculations for all-anti alkyl segments. Part of the X-ray single-crystal structure with non-parallel aromatic rings and H-bonding is shown in the inset in (a). “ssb”: spinning sideband.

FIG. 12 shows the results of determining ¹H chemical shifts in 2D solid-state ¹H-¹³C HetCor NMR spectra (sp²-hybridized-carbon regions), wherein FIG. 12(a) shows a reference diacetylene with 0.2 ms of cross polarization, FIG. 12(b) compound 1 with 0.4 ms of CP (across a couple of bond distances), and FIG. 12(c) compound 2 with 0.4 ms of CP. Arrows highlight the high ¹H chemical shifts observed most prominently at the amide C═O, assigned to H-bonded N—H, and the unusually low aromatic proton chemical shifts relative to values in solution (purple bar), indicative of aromatic CH-π interactions.

FIG. 13 shows the results of determining ¹H chemical shifts in 2D solid-state ¹H-¹³C HetCor NMR spectrum of poly(ethylene terephthalate) (PET) fibers, with 0.072 ms cross polarization. With parallel stacked rings, a high aromatic ¹H chemical shift is observed.

FIG. 14 shows simulated chemical shifts of the diacetylene diazobenzene for: FIG. 14(a) the all-anti X-ray crystal structure and FIG. 14(b) a hypothetical structure with a gauche-anti-gauche zig-zag conformation as shown in FIG. 14(c). The chemical-shift patterns near 30 ppm are clearly distinct.

FIG. 15 shows the results of determining ¹H chemical shifts in 2D solid-state ¹H-¹³C HetCor NMR spectra (alkyl regions) with 0.072 ms of CP for: FIG. 15(a) compound 1 and FIG. 15(b) compound 2. Red arrows highlight unusually low aromatic proton chemical shifts relative to values in solution (purple ‘x’ marks), indicative of alkyl CH—π interactions.

FIG. 16 shows the results of probing phenyl-alkyl proximity in two-dimensional solid-state ¹H-¹³C HetCor NMR spectra (sp²-hybridized-carbon regions) with 1.1 ms of cross polarization (across several bond distances) of: FIG. 16(a) reference diacetylene, FIG. 16(b) compound 1 (including 20% of a minor crystal form), and FIG. 16(c) compound 2. Alkyl-to-phenyl cross-peaks in FIG. 16(c) are highlighted by red arrows.

FIG. 17A shows VT-NMR of compound 1 at −40° C., −15° C., and −5° C.

FIG. 17B VT-NMR of compound 2 at −5° C., 10° C., and 25° C. The red dash lines mark the significant chemical shift of protons.

FIG. 18 shows DSC plots illustrating thermal properties of E (top curve), thermal isomerization from Z-to-E (middle curve), and Z (bottom curve) upon heating and cooling for compounds 1-4. The following thermal parameters are labeled in plots: crystallization temperature (T_{c}), melting temperature (Tₘ), cold-crystallization temperature (T_{cc}), and onset temperature of Z-to-E thermal isomerization (Tᵢₛₒ). The first heating or cooling curve is shown in red, and the following cooling or heating curve is in grey.

FIG. 19 shows XRD patterns of E (black) and Z (red) isomers of compounds 1-4.

FIG. 20 shows UV-Vis absorption spectra of compounds 3 and 4 in DCM solutions.

FIG. 21 shows the results of the process of screening the best solvent candidates for reversible solubility changes of compound 1. Upon irradiation of 340 nm and 430 nm, MeCN and toluene were found to be the ideal solvent candidates for the compound.

FIG. 22 shows the results of the process of screening the best solvent candidates for reversible solubility changes of compound 2. Upon irradiation of 340 nm and 430 nm, MeOH and EtOAc were found to be the ideal solvent candidates for the compound.

FIG. 23 shows the results of the process of screening the best solvent candidates for reversible solubility changes of compound 3. Upon irradiation of 340 nm and 430 nm, MeOH and acetone were found to be the ideal solvent candidates for the compound.

FIG. 24 shows the results of the process of screening the best solvent for reversible solubility changes of compound 4. Upon irradiation of 340 nm and 430 nm, no solvent is ideal for the compound.

FIG. 25 shows optical images of 1-Z and 2-Z solutions under 430 nm irradiation over time.

FIG. 26 shows Eyring-Polanyi plots of thermal isomerization Z-to-E of compounds 1 and 2 measured in MeCN and MeOH, respectively.

FIG. 27 shows the change in the concentration of Z isomer (%) over time in the Michael addition reaction mixtures with soluble Z of compound 1 (left) and compound 2 (right).

FIG. 28 shows (a) rate constants of Michael addition catalyzed by compound 1 of soluble Z (red) and insoluble E (orange); (b) product yield catalyzed by compound 2 of soluble Z (red) and insoluble E (orange) over time. Reaction in pure MeOH as the reference (grey); and (c) rate constants of Michael addition catalyzed by compound 2 of soluble Z (red) and insoluble E(orange).

FIG. 29A shows a ¹H NMR of compound 1 mixed with each reactant or product of Michael addition reaction. Red dashed lines and numbers mark the significant chemical shift changes of proton signals. The concentration of reactant or product is 10-fold higher than that of catalyst, so some NMR peaks corresponding to the reactant or product exhibit high intensities above the range of interest. The black dashed lines indicate those peaks that are omitted to better illustrate the peak shifts from compound 1.

FIG. 29B shows a ¹H NMR of compound 2 mixed with each reactant or product of Michael addition reaction. Red dashed lines and numbers mark the significant chemical shift changes of proton signals. The concentration of reactant or product is 10-fold higher than that of catalyst, so some NMR peaks corresponding to the reactant or product exhibit high intensities above the range of interest. The black dashed lines indicate those peaks that are omitted to better illustrate the peak shifts from compound 2.

FIG. 30 shows absorbance of compound 1 at 241 nm (left) and compound 2 at 280 nm (right) upon long-term 340 nm irradiation at 20° C. (˜1.7×10−5 M in MeCN and MeOH, respectively).

FIG. 31 shows the recovery rate of compound 2 (green) and percentage of product yield (blue) in three cycles of a Michael addition reaction.

FIG. 32 shows (a) rate constants of a Henry reaction catalyzed by compound 1 of soluble Z (red), insoluble E with 340 nm irradiation (blue), and insoluble E(orange). (b) Monitoring the product yield catalyzed by compound 2 of soluble Z (red), insoluble E with 340 nm irradiation (blue), and insoluble E (orange). Reaction in pure MeOH as the reference (grey). (c) Rate constants of Henry reaction catalyzed by compound 2 of soluble Z (red), insoluble E with 340 nm irradiation (blue), and insoluble E (orange).

FIG. 33 shows the ratio changes of Z isomer in the Henry reaction mixtures with soluble Z and insoluble E isomers with 340 nm irradiation of (a) compound 1 and (b) compound 2.

FIG. 34 shows (a) a ¹H NMR of compound 1 mixed with each reactant or product of Henry reaction; (b) a ¹H NMR of β-nitroalcohol (product) mixed with 10 mol % compound 1; and (c) a ¹H NMR of compound 2 mixed with each reactant or product of Henry reaction. Red dashed lines and numbers mark the significant chemical shift changes of proton signals. The concentration of reactant or product is 10-fold higher than that of catalyst, so some NMR peaks corresponding to the reactant or product exhibit high intensities above the range of interest. The black dashed lines indicate those peaks that are omitted to better illustrate the peak shifts from compounds 1 and 2.

FIG. 35A and FIG. 35B are ¹H NMR spectra of the 3^{rd} cycle of Henry reaction after 430 nm irradiation. The ratios of E isomers of compound 1 (FIG. 35A)) and compound 2 (FIG. 35B) determined by the peaks of E and Z isomers.

FIG. 36 shows the recovery rate of compound 2 (green) and percentage of product yield (blue) in three cycles for the Henry reaction.

### DETAILED DESCRIPTION

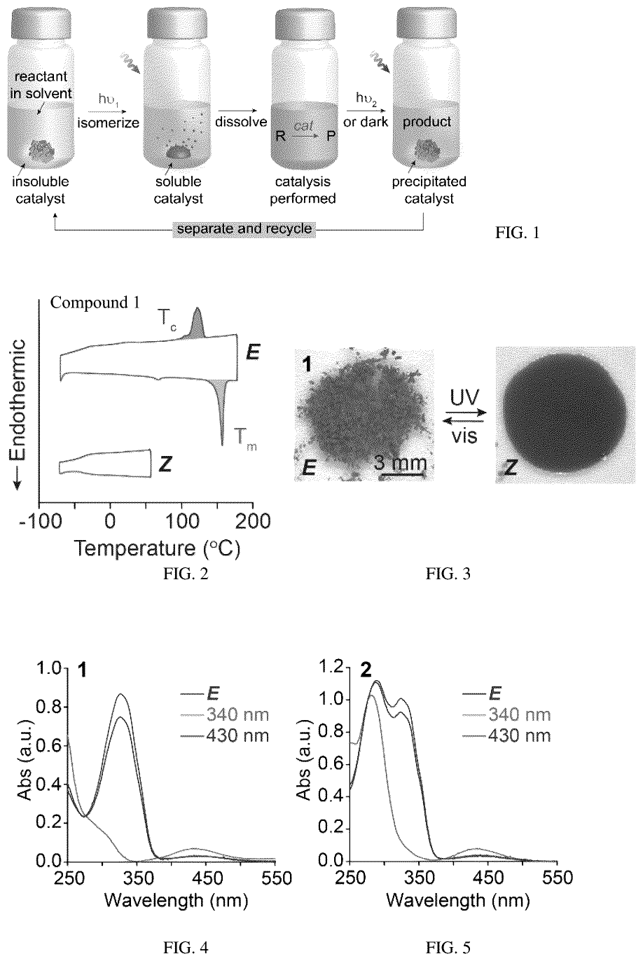

This invention addresses a critical challenge of recovering and recycling homogeneous organocatalysts by designing photoswitchable catalyst structures that display a reversible solubility change in response to light. For example, as illustrated in FIG. 1, an initially-insoluble catalyst is UV-switched to a soluble isomeric state, which catalyzes the reaction, then back-isomerized to the insoluble state upon completion of the reaction, to be filtered and recycled. The molecular design principles allow for a significant solubility change, which can be over 10 times between the isomeric state. In addition, 87% recovery by the light-induced precipitation, and multiple rounds of catalyst recycling can be achieved. The invention accordingly allows the development of highly-recyclable, homogeneous catalysts, which are important for the synthesis of critical compounds in various industries. The invention can further significantly reduce the environmental impact of catalysts used in these industries.

A variety of photoswitches with diverse optical properties have been reported, e.g., spiropyran/merocyanine, diarylethene, azobenzene, arylazopyrazole, azothiophene, hydrazone, Stenhouse adduct, norbornadiene/quadricyclane (NBD/QC), and dihydroazulene/vinylheptafulvene (DHA/VHF), which provide a wide collection of photoswitch units available to tailor the catalyst structure to various reaction conditions. The method of optical stimulation used to trigger these photoswitches presents a unique opportunity for regulating catalytic activity due to its non-invasive nature and high spaciotemporal resolution. In optically-controlled biphasic systems, in an aspect, an insoluble solid catalyst undergoes a light-mediated phase change into a liquid and dissolves into an organic solvent to catalyze and complete the desired reaction. Then the “light-activated” catalyst can be precipitated in response to another wavelength of light, filtered, and recycled, enabling sustainable catalysis at a reduced cost and environmental impact.

Photo-responsive phase change materials that incorporate azobenzene or azoheteroarene photoswitches therefor allow a new class of photo-controlled phase change catalysts to be developed, with the key feature of large solubility changes between two isomeric forms. In an aspect, for some photo-responsive phase change materials, a reversible solid-liquid transition is prompted by the optically-triggered structural change of switches between a planar (E) and a nonplanar (Z) isomer. Without being bound by theory, it is believed that the sterically-bulky Z configuration effectively disrupts π-interactions between the aromatic groups of planar E isomers in a crystalline phase, resulting in the formation of a less solid phase that can be a liquid phase. The reverse isomerization (Z to E) recovers the planar isomeric structures and crystalline packing between molecules. It is further hypothesized that photo-isomerization will considerably alter the solubility of molecules in organic solvents, thus controlling catalytic activities. However, the concept of phase-dependent catalysis is an area of great potential that has not yet been fully explored in photoswitch chemistry.

The photoresponsive phase change catalysts described herein are accordingly organocatalysts that photoswitch between a first isomer and a second isomer of differing solubilities in an organic solvent. Such organocatalysts include compounds of Formula I that have groups that affect the solubility of the catalyst in an organic solvent; photoactivated groups that change, for example isomerize or cyclize in response to light; and at least one catalytic site for catalyzing a reaction.

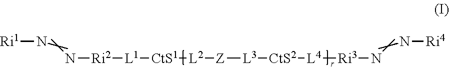

In Formula (I), Ri¹, Ri², Ri³, and Ri⁴ are each independently an aryl or heteroaryl 5- or 6-membered ring optionally substituted with a C₁₋₆ alkyl, C₁₋₆ alkoxy, C₁₋₆ alkylthio, halogen, cyano, halogenated C₁₋₆ alkyl, halogenated C₁₋₆ alkoxy, or di(C₁₋₆ alkyl)amino. In an aspect in Formula (I), Ri¹, Ri², Ri³, and Ri⁴ are each independently phenyl, imidazolyl, oxazolyl, isoxazolyl, thiazolyl, or isothiazolyl. In another aspect in Formula (I), Ri¹, Ri², Ri³, and Ri⁴ are each the same ring, or are each the same ring and are phenyl.

In an aspect in Formula (I), each of Ri¹, Ri², Ri³, and Ri⁴ are unsubstituted. In another aspect at least one substituent is present on each of Ri¹, Ri², Ri³, and Ri⁴, where each substituent may be the same or different. When present, each substituent on Ri¹, Ri², Ri³, and Ri⁴ are preferably selected to adjust the solubility of the organocatalyst of Formula (I), for example to increase solubility in an organic solvent. Preferred substituents of this type include halogen, C₁₋₃ alkoxy, or halogenated C₁₋₈ alkyl. The halogen of the halogenated C₁₋₈ alkyl (and the halogenated C₁₋₈ alkoxy) can be chlorine, bromine, or fluorine, and the alkyl or alkoxy group can have one or more halogens present, or be perhalogenated, i.e., a group of the formula —(CX₂)ₙCX₃ or —(CX₂)ₙOCX₃ wherein n is 1 to 8 and X is a halogen, preferably chlorine, bromine, or fluorine. In an aspect, C₁₋₃ alkoxy, or halogenated C₁₋₃ alkyl, such as halomethyl, dihalomethyl, and trihalomethyl.

Further in Formula (I), each substituent of Ri¹, Ri², Ri³, and Ri⁴ can be in an ortho position to the azo group, and can be independently halogen, C₁₋₆ alkoxy, C₁₋₆ alkylthio, halomethyl, dihalomethyl, trihalomethyl, or di(C₁₋₆ alkyl)amino. In an aspect in Formula (I), each substituent of Ri¹, Ri², Ri³, and Ri⁴ is independently the same or different halogen. In another aspect in Formula (I), each of Ri¹, Ri², Ri³, and Ri⁴ is the same and is F, Cl, or Br, preferably F or Cl. In an aspect in Formula (I), two of Ri¹, Ri², Ri³, and Ri⁴ are F, and the other two of Ri¹, Ri², Ri³, and Ri⁴ are Cl. In another aspect in Formula (I), each of Ri¹, Ri², Ri³, and Ri⁴ is independently ethoxy or methoxy, or each of Ri¹, Ri², Ri³, and Ri⁴ is the same and is ethoxy or methoxy.

Still further in Formula (I), each L¹, L², L³, and L⁴ is independently a bond or a C₁. ₃₀ linking group optionally including a heteroatom. In an aspect each of L¹, L², L³, and L⁴ is the same. In another aspect, two of L¹, L², L³, and L⁴ are the same (e.g., a bond), and two are different (e.g., a group). L¹, L², L³, and L⁴, or at least two of L¹, L², L³, and L⁴ are preferably selected to decrease the solubility of the organocatalyst in the organic solvent. Thus, the solubility of an organocatalyst in the organic solvent can be adjusted by the appropriate selection of Ri¹, Ri², Ri³, and Ri⁴ ring types, substituents on Ri¹, Ri², Ri³, and Ri⁴, and L¹, L², L³, and L⁴.

In an aspect, L¹, L², L³, and L⁴ can be hydrogen-bonding groups having from 1 to 30 carbon atoms and that contain a heteroatom, such as O, N, P, Si, or S, or a combination thereof, preferably N, O, P, or a combination thereof. Exemplary hydrogen bonding groups include functional groups such as —C(O)O—, —C(O)NH—, —NHC(O)NH—, or —C(O)NHC(O)—, preferably —C(O)O— or —C(O)NH—. In an aspect in Formula (I), L¹, L², L³, and L⁴ can include a heterocycle, i.e., a carbocyclic ring group that includes one or more saturated, unsaturated, or aromatic rings, in which a ring member (e.g., one, two or three ring members) is a heteroatom. In a C₃₋₃₀ heterocycle, the total number of ring carbon atoms is from 3 to 30, with remaining ring atoms being heteroatoms. Multiple rings, if present, may be pendent, spiro or fused. Exemplary heterocycle groups include cyclopropyl, cyclohexyl, cyclohexenyl, cycloalkynyl, thienyl, benzo[b]thienyl, naphtho[2,3-b]thienyl, thianthrenyl, furyl, benzofuranyl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolinyl, quinolinyl, phthalazinyl, naphthyridinyl, cinolinyl, quinazolinyl, pterridinyl, 4H-oxazolyl, oxazolyl, β-oxazoline, phenanthridinyl, acridinyl, pyrimidinyl, phenanthrolinyl (phenanthrolinyl), phenazinyl, thiazolyl, isothiazolyl, phenothiazolyl, isoxazolyl, furazinyl and phenoxazinyl. When L¹, L², L³, or L⁴ is a heterocycle, the group can further include C₁₋₆ alkyl groups or other functional groups to incorporate the linker into the organocatalyst.

Yet further in the organocatalyst of Formula (I), CtS¹ and CtS² are each independently catalytic moieties, i.e., groups that can catalyze the formation of a product from a reactant. A wide variety of catalytic moieties are known in the art and can be incorporated into the organocatalysts, for example various heteroatoms (e.g., N), groups containing a heteroatom (e.g., B, N, O, S, P, Si, or S), and metal-containing complexes. As is known, a tertiary amino group or a secondary amino containing an active hydrogen can act as a catalyst. Other groups containing a heteroatom, such as the heterocycles described above, can act as a catalyst. In still other embodiments, the catalyst moiety can include groups that provide both that are favorable for self-assembly (e.g., via hydrogen-bonding) and catalytic activity, for example, groups such as thioureas, squaramides, and the like. Other catalytic motifs include photo-organocatalytic moieties bearing aromatic groups, such as 10-phenylphenothiazine and thiocarbamate-functionalized indole, which can also contribute to self-assembly via π-interactions. The metal-containing complexes can include a transition metal such as iron, platinum, chromium, copper, palladium, and the like in various oxidation states.

Further in Formula (I), Z is a moiety that affects the phase-change properties of the organocatalyst, the solubility of the organocatalyst, or both. In an aspect, Z contributes to the crystallinity of a first isomer of the organocatalyst, i.e., increases insolubility in the organic solvent. Exemplary Z moieties include phenyl, naphthyl, anthracenyl, benzo[a]anthracenyl, benzo[a]pyrenyl, perylenyl, chrysyl, fluoranthrenyl, phenanthrenyl, benzo[l]fluorenyl, biphenyl, triphenyl, dialkyne. trialkyne, a combination thereof.

Finally in Formula (I), r is an integer from 0 to 4. In an aspect, r is 0 and a catalytic moiety is bound to one or more of the rings, one or more linking groups, or a combination of a ring and a linking group, for example an adjacent ring and linking group such as Ri² and L¹, or Ri³ and L⁴, or a combination thereof. The catalytic moiety can be the same or different. In an aspect, r is 1. In another aspect, r is 2.

Exemplary organocatalysts of Formula (I) wherein r is 0 are of Formula (II)

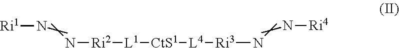

wherein Ri¹, Ri², Ri³, and Ri⁴, L¹ and L⁴, and CtS¹ are the same as described above.

In an aspect in Formula (II) Ri¹, Ri², Ri³, and Ri⁴ are each independently an aryl or heteroaryl 5- or 6-membered ring optionally substituted with one or more of C₁₋₆ alkyl, C₁₋₆ alkoxy, C₁₋₆ alkylthio, halogen, trihalomethyl, halo, cyano, halogenated C₁₋₆ alkyl, halogenated C₁₋₆ alkoxy, or di(C₁₋₆ alkyl)amino; L¹ and L⁴ is each independently a bond or a C₁₋₃₀ linking group; and CtS¹ is a catalytic moiety.

In another aspect in Formula (II), Ri¹, Ri², Ri³, and Ri⁴ are each independently an phenyl, imidazolyl, oxazolyl, isoxazolyl, thiazolyl, or isothiazolyl, or Ri¹, Ri², Ri³, and Ri⁴ are each the same ring, or are each the same ring and are phenyl, wherein Ri¹, Ri², Ri³, and Ri⁴ are optionally substituted with a C₁₋₃ alkyl, C₁₋₆ alkoxy, halo, cyano, halo, halogenated C₁₋₆ alkyl, halogenated C₁₋₆ alkoxy, or di(C₁₋₆ alkyl)amino; and L¹ and L⁴ is each independently a bond or a C₁₋₁₈ linking group optionally including a heteroatom.

In another aspect in Formula (II), Ri¹, Ri², Ri³, and Ri⁴ are each independently a phenyl optionally substituted with a C₁₋₃ alkyl, C₁₋₆ alkoxy, halo, or halogenated C₁₋₆ alkyl; and L¹ and L⁴ is each independently a bond.

An exemplary organocatalyst of this type is of Formula (IIa)

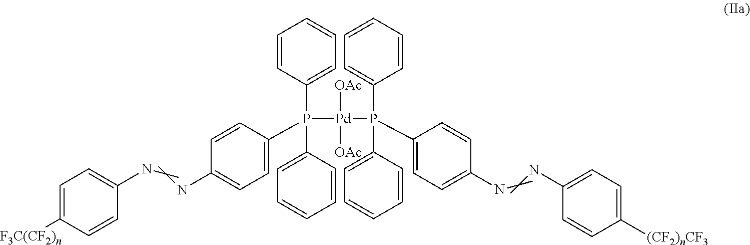

wherein OAc is —OC(═O)CH₃.

In another aspect, exemplary organocatalysts of Formula (I) wherein r is 2 are of Formula (III)

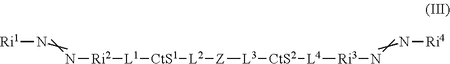

wherein Ri¹, Ri², Ri³, and Ri⁴, L¹ and L⁴, and CtS¹ and CtS² are the same as described above.

In an aspect in Formula (III), Ri¹, Ri², Ri³, and Ri⁴ are each independently an aryl or heteroaryl 5- or 6-membered ring optionally substituted with one or more of C₁₋₆ alkyl, C₁₋₆ alkoxy, C₁₋₆ alkylthio, halogen, trihalomethyl, halo, cyano, halogenated C₁₋₆ alkyl, halogenated C₁₋₆ alkoxy, or di(C₁₋₆ alkyl)amino; L¹, L², L³, and L⁴ is each independently a bond or a C₁₋₃₀ linking group; and CtS¹ and CtS² are a catalytic moiety that can be the same or different.

In another aspect in Formula (III), Ri¹, Ri², Ri³, and Ri⁴ are each independently a phenyl, imidazolyl, oxazolyl, isoxazolyl, thiazolyl, or isothiazolyl, or Ri¹, Ri², Ri³, and Ri⁴ are each the same ring, or are each the same ring and are phenyl, wherein Ri¹, Ri², Ri³, and Ri⁴ are optionally substituted with a C₁₋₃ alkyl, C₁₋₆ alkoxy, halo, cyano, halo, halogenated C₁₋₆ alkyl, halogenated C₁₋₆ alkoxy, or di(C₁₋₆ alkyl)amino; and L¹, L², L³, and L⁴ are each independently a bond or a C₁₋₁₈ linking group optionally including a heteroatom.

In another aspect in Formula (II), Ri¹, Ri², Ri³, and Ri⁴ are each independently a phenyl optionally substituted with a C₁₋₃ alkyl, C₁₋₆ alkoxy, halo, halo, or halogenated C₁₋₆ alkyl; and L¹, L², L³, and L⁴ are each independently C₁₋₁₈ linking group without a heteroatom, or a linear or branched (C₁₋₁₈ alkyl) group including a heteroatom, preferably including a group of the formula —C(O)O—, —C(O)NH—, —NHC(O)NH—, or —C(O)NHC(O)—, preferably a linear or branched (C₁₋₆ alkyl) group including C(O)O—, or —C(O)NH—, more preferably a linear or branched (C₁₋₆ alkyl) group of the formula —C(O)NH—. In an aspect, each of L¹ and L⁴ is a linear or branched (C₁₋₆ alkyl) group and each of L³ and L⁴ include a (C₁₋₆ alkyl) group including —C(O)NH—.

An exemplary organocatalyst of this type is of Formula (IIIa)

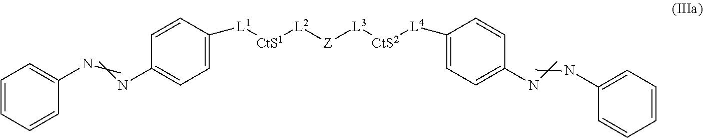

wherein in Formula (IIIa), L¹, L², L³, and L⁴ is each independently a bond or a C₁₋₃₀ linking group; Z is a group that increases crystallinity of the organocatalyst; and CtS¹ and CtS² are a catalytic moiety as described above that can be the same or different. Alternatively, L¹ and L⁴ can be the same group (preferably without a heteroatom), and L² and L³ can be the same group (preferably a hydrogen-bonding group), that differs from L¹ and L⁴. Thus, the solubility of an organocatalyst in the organic solvent can be adjusted by the appropriate selection of Ri¹, Ri², Ri³, and Ri⁴ ring types, substituents on Ri¹, Ri², Ri³, and Ri⁴, and L¹, L², L³, and L⁴.

In another aspect in Formula (IIIa), Z is phenyl, anthracenyl, biphenyl, triphenyl, dialkyne. trialkyne, or a combination thereof; and L¹, L², L³, and L⁴ are each independently a bond or a C₁₋₁₈ linking group including a heteroatom. Alternatively in this aspect, L¹ and L⁴ can be the same group (preferably without a heteroatom), and L² and L³ can be the same group (preferably a hydrogen-bonding group), that differs from L¹ and L⁴.

In another aspect in Formula (IIIa), Z is phenyl, anthracenyl, biphenyl, dialkyne, or trialkyne; at least two of L¹, L², L³, and L⁴ are each independently a C₁₋₁₈ linking group optionally including a heteroatom, and at least two of L¹, L², L³, and L⁴ are each independently a linking group of the formula (C₁₋₁₂ alkylene)or (C₁₋₆ alkylene) and further including —C(O)O—, —C(O)NH—, —NHC(O)NH—, or —C(O)NHC(O)—, preferably —C(O)O—, or —C(O)NH—, more preferably a group of the formula —C(O)NH—. Alternatively, L¹ and L⁴ can be the same C₁₋₁₂ or C₁₋₆ hydrocarbon with no heteroatom, and L² and L³ can be the same group (preferably a hydrogen-bonding group), that differs from L¹ and L⁴, preferably a group of the formula (C₁₋₁₂ alkylene)or (C₁₋₆ alkylene) and further including —C(O)O—, —C(O)NH—, —NHC(O)NH—, or —C(O)NHC(O)—, preferably —C(O)O—, or —C(O)NH—, more preferably a group of the formula —C(O)NH—. In an aspect, each of L¹ and L⁴ is a linear or branched (C₁₋₆ alkyl) group and each of L³ and L⁴ include a linear or branched (C₁₋₆ alkyl) group including —C(O)NH—.

Exemplary compounds of Formula (IIIa) are compound 1, compound 2, compound 3, and compound 4.

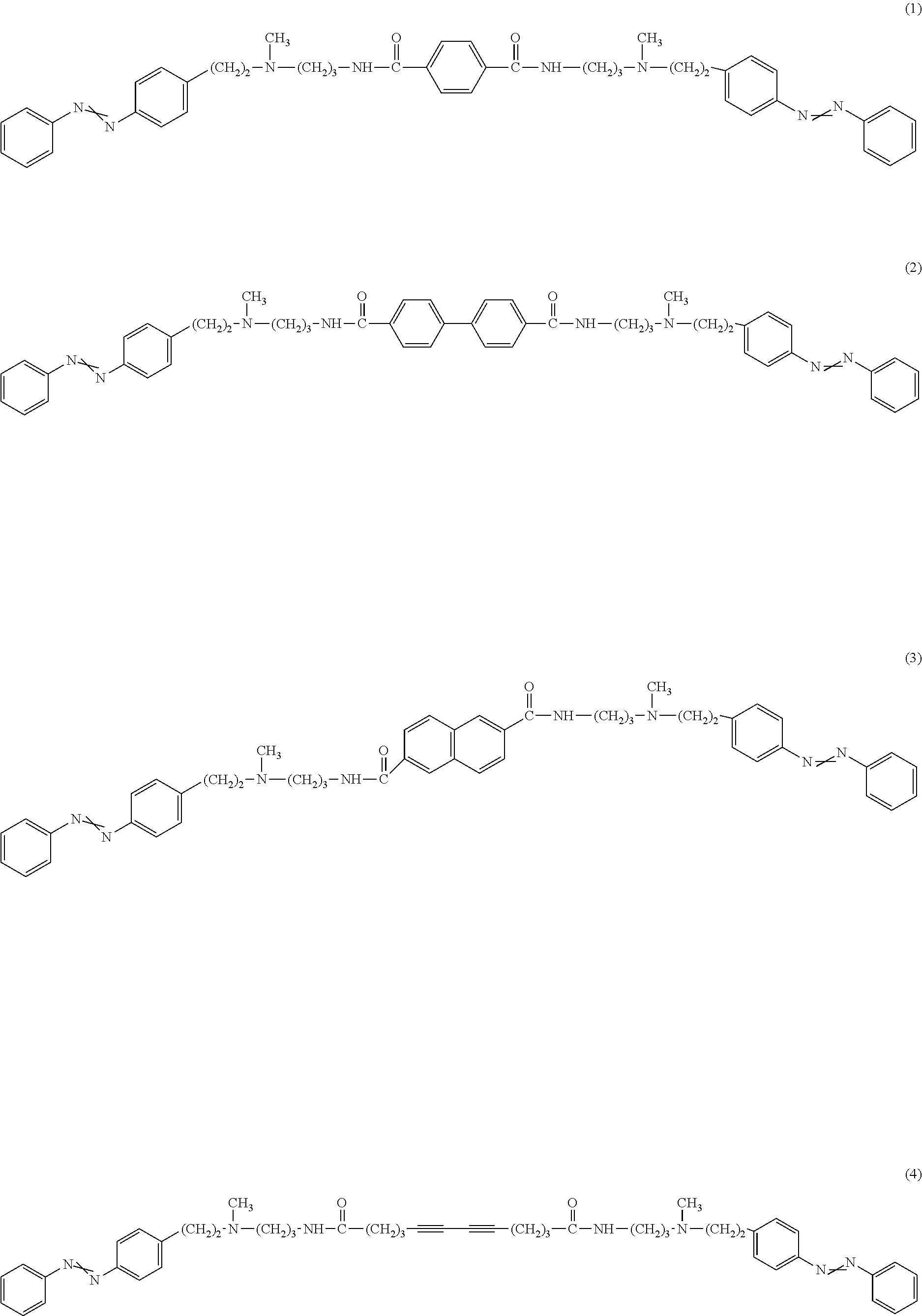

Another exemplary compound of Formula (IIIa) is compound 15.

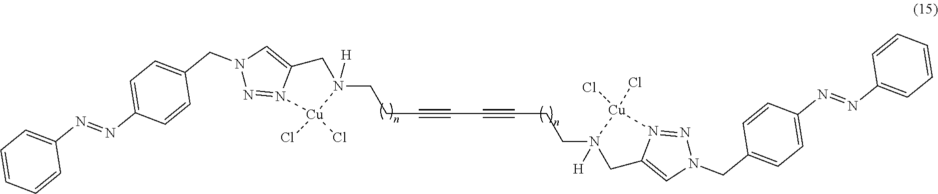

In an embodiment a composition comprises, consists of, or consists essentially of, a compound of Formulas (I), (II), or (III). In the composition, the compound of Formulas (I), (II), or (III) can be present in the form of a Z-isomer, an E-isomer, or a combination thereof. The compositions can further comprise other components, provided that such components do not substantially adversely affect the desired properties of the composition, for example transparency to sunlight or visible light. Other components can be, for example, trace amounts of organic solvents as well as antioxidants, thermal stabilizers, mold release agents, or the like. The composition can consist of a single compound of Formulas (I), (II), or (III) in a substantially pure form, such as at least about 95% pure, at least about 97% pure, at least about 98% pure, or at least about 99% pure. This is without regard to the (E)/(Z) form of the compound.

The compounds of Formulas (I), (II), and (III) have a number of advantageous properties, including the capability of transforming from the E-isomer to the Z-isomer, or from the Z-isomer to the E-isomer upon irradiation with the appropriate wavelength of light. This isomeric transformation results in a phase change of the compounds, from more crystalline (the E-isomer) to less crystalline (the Z-isomer). Generally, the more crystalline forms can be more solid and the less crystalline forms can be less solid. Here, however, the more important property is that the E-isomers (the more crystalline forms), can have a lower solubility in an organic solvent than that of the Z-isomers (the less crystalline forms), which can have a greater solubility in the organic solvent. Appropriate selection of the Ri¹, Ri², Ri³, and Ri⁴ groups, their optional substituents, L¹, L², L³, and L⁴, Z, and CtS¹ and CtS² can be used to tune the solubility of the compounds in different organic solvents. This change in solubility allows use of the compounds of Formula III not just as organocatalysts, but as recoverable organocatalysts.

As stated above, the compounds of Formulas(I), (II), and (IIII) can be used as a homogenous organocatalyst, in particular a photoswitchable homogenous organocatalyst, and even more particularly as a recyclable photoswitchable homogeneous catalyst.

Accordingly, a method of catalyzing a reaction includes providing an organic reaction medium comprising the compound of Formulas (I), (II), or (III) having a first phase having a lower solubility in an organic solvent than a second phase; and a reactant subject to a catalytic transformation by the compound of Formulas (I), (II), or (III); irradiating the reaction medium with a first wavelength of light (e.g., in the ultraviolet light range) to effect a phase change of the compound of Formulas (I), (II), or (III) to a second phase having a second, higher solubility in the organic solvent; and catalyzing the transformation of the reactant to a product.

In an advantageous feature, the second phase of the compound of Formulas (I), (II), or (III) can be irradiated with light having a second wavelength (e.g., in the visible light range) to convert the second phase to the first phase. At least two (for example, three, four, five, six, seven, eight, nine, ten, or even more than ten) cycles of the providing, irradiating with the first wavelength, catalyzing, and irradiating with the second wavelength can be performed.

Optionally, after irradiating with the second wavelength of light, the first phase of the compound of Formulas (I), (II), or (III) can be isolated from the reaction medium. In this aspect, at least two (for example, three, four, five, six, seven, eight, nine, ten, or even more than ten) cycles of the providing, irradiating with the first wavelength, catalyzing, irradiating with the second wavelength, and separating can be performed.

Another method of catalyzing a reaction includes providing an organic reaction medium comprising the compound of Formulas (I), (II), or (III) having the second phase having a higher solubility in an organic solvent than the first phase, and a reactant subject to a catalytic transformation by the compound of Formulas (I), (II), or (III); and catalyzing the transformation of the reactant to a product. As above, in an advantageous feature, the second phase of the compound of Formulas (I), (II), or (III) can be irradiated with light having the second wavelength (e.g., in the visible light range) to convert the second phase to the first phase; and then the first phase irradiated with light having the first wavelength (e.g., in the ultraviolet range) to convert the first phase of the compound of Formulas (I), (II), or (III) to the second phase. Additional reactant can be added to the reaction medium, before or after the irradiating with light having the first wavelength (e.g., in the ultraviolet range) to convert the first phase of the compound of Formulas (I), (II), or (III) to the second phase. Preferably, additional reactant is added after the irradiating with light having the second wavelength (e.g., in the visible light range) to convert the second phase to the first phase At least two (for example, three, four, five, six, seven, eight, nine, ten, or even more than ten) cycles of the providing, catalyzing, irradiating with the second wavelength, adding additional reactant, and irradiating with the first wavelength can be performed.

Optionally, after irradiating with the light having a second wavelength, the first phase of the compound of Formulas (I), (II), or (III) can be isolated from the reaction medium before irradiating with the second wavelength light. In this aspect, at least two (for example, three, four, five, six, seven, eight, nine, ten, or even more than ten) cycles of the providing, catalyzing, irradiating with the second wavelength, separating, and irradiating with the first wavelength can be performed.

The phase changes of the compound of Formulas (I), (II), or (III) can be induced at a wide variety of temperatures depending on the particular compound used, for example, from −25° C. to 150° C. In an aspect, the temperature is the same as, or within ±50° C. of the reaction temperature used for catalysis. The phase change can be induced by exposing the isomer to light in the ultraviolet (UV) or visible (Vis) spectrum depending on the particular compound used, which for convenience can be defined as 100-400 nm (UV) and 401-700 nm (Vis).

The compound of Formulas (I), (II), or (III) can be used for the catalysis of a wide variety of reactions, for example, hydroformylation, hydroboration, epoxidation of alkenes, a Michael reaction, a Henry reaction a Mannich reaction, a Diels-Alder reaction, or the like. Reaction conditions for catalysis are known for use in the art for the particular reaction being conducted. The organic reaction medium includes an organic solvent, and other adjuvants as known in the art, for example co-solvents, co-catalysts, and the like.

A wide variety of organic solvents can be used, for example aliphatic hydrocarbons such as C5-30 straight, branched-chain, or cyclic aliphatic hydrocarbons, e.g., pentane, hexane, heptane, cyclohexane, methylcyclohexane, mineral oil (also referred to as liquid petrolatum or liquid paraffin), mineral spirits (also referred to as ligroin or petroleum spirits), aromatic hydrocarbons, for example benzene, xylene, decaline, naphthalene, C₁₋₈ alkyl derivatives of benzene, and C₁₋₈ alkyl derivatives of naphthalene, specifically toluene, xylene (o, m, or p), cumene, ethyl benzene, mesitylene, durene, sec-amylbenzene, n-butylbenzene, naphthalene, and methyl naphthalene; aldehydes such as acetaldehyde, benzaldehyde, and nitrobenzaldehyde; straight-chain and cyclic ethers, such as diethyl ether, diisopropyl ether, methyl tert-butyl ether (MTBE), methyl tert-amyl ether, dioxane, tetrahydrofuran, 1,2-dimethoxyethane, 1,2-diethoxyethane and anisole; nitrile-containing compounds such as acetonitrile, propionitrile, n- or isobutyronitrile or benzonitrile; organic sulfur-containing compounds such as sulfoxides, for example dimethyl sulfoxide (DMSO); chlorinated solvents, for example chlorobenzene, dichlorobenzene, and chlorinated C₁₋₆ aliphatic compounds such as allyl chloride, carbon tetrachloride, chloroform, 1,1-dichloroethane, dichloroethyl ether, 1,2-dichloroethylene, dichloroisopropyl ether, ethyl chloride, ethylene dichloride, isopropyl chloride, methyl chloride, perchloroethylene, propylene dichloride, 1,1,2-trichloroethane, trichloroethylene 1,2,3 trichloropropene, and methylene chloride (dichloromethane, or DCM); alcohols, for example amyl alcohol, n-butanol, 3-butoxyethyl-2-propanol, benzyl alcohol, benzyloxyethanol, diethoxyethanol, diisobutyl carbinol, dimethyl heptanol, ethanol, 2-ethylhexanol, ethylene glycol, glycerin, 1-hexanol, isobutanol, isopropanol, methanol, methyl amyl alcohol, 2-methyl-1-butanol, 1-pentanol, 1-propanol, propylene glycol, and 2,2,4-trimethyl-1,3-pentanediol monoisobutyrate; glycol ethers, for example diethylene glycol methyl ether, diethylene glycol mono-n-butyl ether, diethylene glycol monoethyl ether, diethylene glycol monomethyl ether, diethylene glycol monopropyl ether, diethylene glycol n-butyl ether acetate, dipropylene glycol monobutyl ether, dipropylene glycol monomethyl ether, dipropylene glycol monopropyl ether, dipropylene glycol tert-butyl ether, ethylene glycol methyl ether acetate, ethylene glycol monobutyl ether, ethylene glycol monohexyl ether, ethylene glycol monopropyl ether, ethylene glycol n-butyl ether acetate, ethylene glycol phenyl ether propylene glycol monobutyl ether, tetraethylene glycol monobenzyl ether, tetraethylene glycol monophenyl ether, triethylene glycol methyl ether, triethylene glycol monobenzyl ether, triethylene glycol monophenyl ether, tripropylene glycol methyl ether), and tripropylene glycol n-butyl ether; amides such as acetamidophenol, N,N-dimethyl formamide (DMF)N-methylformanilide, or hexamethylene phosphoric triamide, and acetanilide, and cyclic amides such as 1-methyl-2-pyrrolidone (NMP), N-ethyl-2-pyrrolidone, N-isopropyl-2-pyrrolidone, N-cyclohexyl-2-pyrrolidone, 2-hydroxyethyl-2-pyrrolidone, N-dimethylaminopropyl-2-pyrrolidone, vinyl-pyrrolidone, and 2-pyrrolidone; amines such as 2-(2-aminoethoxy)ethanol, 2-acetyl-1-methylpyrrole, 2-amino-2-methyl-1-propanolalkanolamines (e.g., n-butyldiethanolamine, diethanolamine, diisopropanolamine, dimethylethanolamine, ethanolamine, isopropanolamine, methylisopropanolamine, phenyl diethanolamine, and triethanolamine), cyclic amines (e.g., N-methyl pyrrolidine, N-methylpyyrole, morpholine, and oxazolidines), n-butylaminoethanol, diethylaminoethanol, diglycolamine, 2-methylaminoethanol, and trialkylamines (e.g. triethylamine); ketones and cyclic ketones such as acetone, isobutyl heptyl ketone, isophorone, methyl ethyl ketone, methyl isobutyl ketone, diisobutyl ketone, diacetone alcohol, acetophenone, methyl n-amyl ketone, cyclohexanone, and cycloheptanone; dialkyl carbonates such as dimethyl carbonate, diethyl carbonate, dipropyl carbonate, diisopropyl carbonate, and dibutyl carbonate; cyclic carbonates such as propylene carbonate and ethylene carbonate; monoesters such as amyl acetate, benzyl acetate, benzyl benzoate, butyl acetate, ethyl acetate (EtOAc), ethyl propionate, ethyl lactate, isobutyl acetate, isopropyl acetate, n-butyl propionate, n-pentyl propionate, n-propyl acetate, n-propyl propionate, butyl lactate, the C₁₋₄ alkyl esters of C₆₋₂₂ saturated or unsaturated carboxylic acids, such as the methyl ester of C₆₋₁₄ unsaturated fatty acids; the glycerol ester of fatty acids, including those derived from vegetable oils such as linseed, coconut, palm, soybean, cottonseed, groundnut, sunflower, rape, sesame, olive, corn, safflower, palm kernel, castor oil, peanut, fish, lard, mustard seed, poppyseed, turpentine, and tall oil, and ethyl 3-ethoxypropionate; dibasic esters such as dimethyl adipate, dimethyl succinate, dimethyl glutarate, dimethyl malonate, diethyl adipate, diethyl succinate, diethyl glutarate, dibutyl succinate, and dibutyl glutarate; alkoxylated aromatic alcohols, in particular the alkoxylated aromatic alcohols containing at least one aromatic ring per molecule and alkoxylate units of general formula —(CR¹R³—CR²R⁴—O)n—R⁵ wherein R¹, R², R³, and R⁴ are each independently hydrogen or methyl; R⁵ is hydrogen, a C₁₋₆ alkyl, or phenyl; and n is 2-10, wherein the alkoxylate units are attached to the aromatic ring directly or through an ether (oxygen) linkage or an oxymethylene (—CHR⁸O—) linkage, wherein R¹ is hydrogen or C₁₋₄ alkyl. A combination of any one or more of the foregoing can be used.

Specific solvents that can be used include petroleum ether, hexane, heptane, cyclohexane, methylcyclohexane, benzene, toluene, xylene or decalin; benzaldehyde, nitro benzaldehyde; chlorobenzene, dichlorobenzene, dichloromethane, chloroform, carbon tetrachloride, dichloroethane or trichloroethane; diethyl ether, diisopropyl ether, MTBE, methyl tert-amyl ether, dioxane, tetrahydrofuran, 1,2-dimethoxyethane, 1,2-diethoxyethane or anisole; acetonitrile, propionitrile, n- or isobutyronitrile or benzonitrile; DMSO; DMF, N,N-dimethylacetamide, N-methylformanilide, NMP or hexamethylene phosphoric triamide; or a combination thereof. Water can be present provided that it does not form a second phase with the organic solvent.

To illustrate the utility of the organocatalysts as described herein, the central groups of the organocatalyst of Formula (IIIa) were varied among phenyl (compound 1), biphenyl (compound 2), naphthyl (compound 3), and diacetylenyl (compound 4) to fine-tune the intermolecular π-interactions, which facilitate the alignment of molecules in solid state. A comparative diacetylenyl compound (compound c) was also studied.

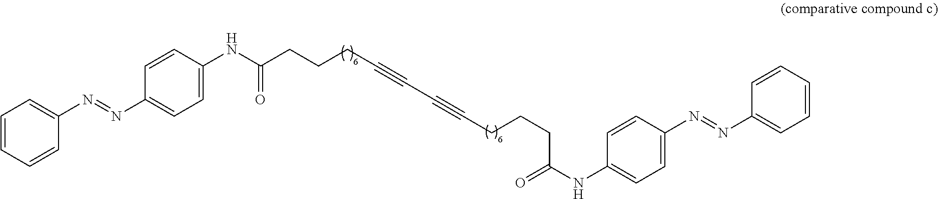

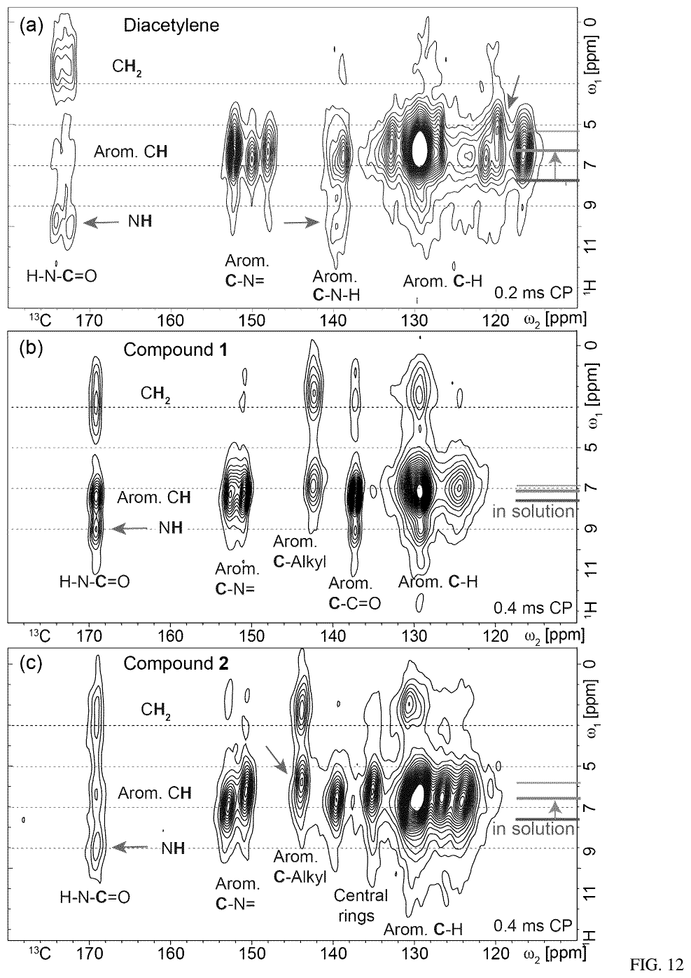

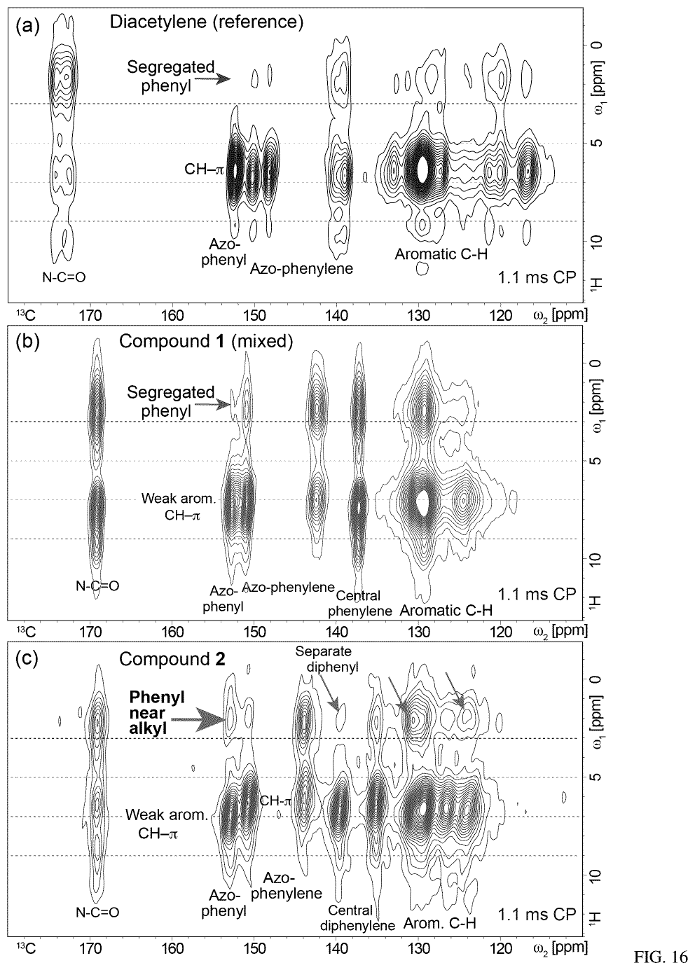

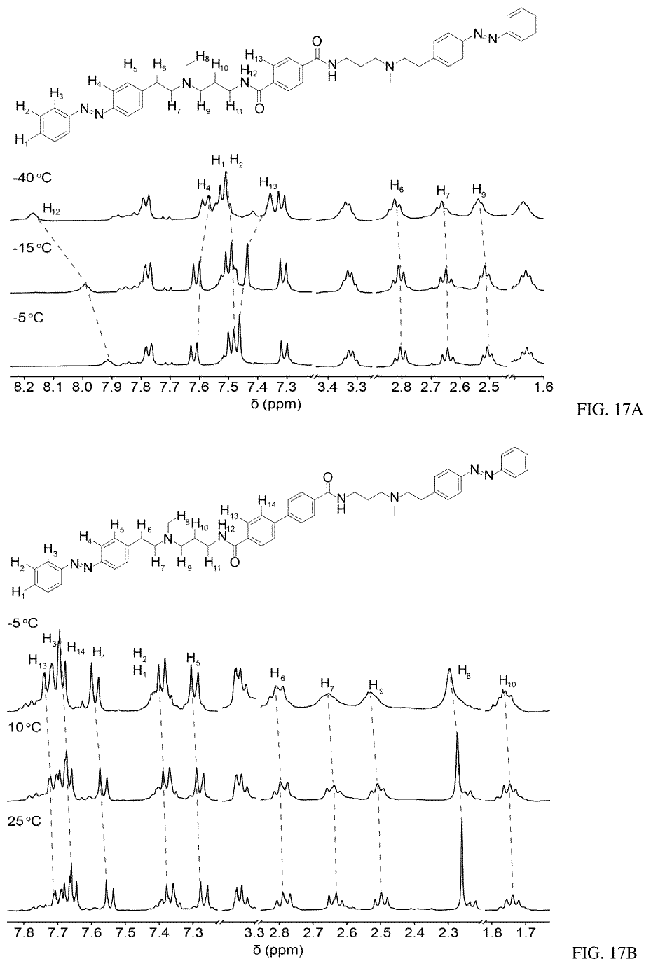

The formation of intermolecular H-bonds between amides and 7-interactions between aromatic moieties were demonstrated in solid-state NMR and solution-state variable-temperature NMR studies. Specifically, compound 1 displays domains of alkyl and aromatic groups that are separated from each other (FIG. 12(b) and FIG. 16(b). compound 2 shows edge-to-face π-interactions among the phenylene rings, which are clearly detected as the significant chemical shift changes of the deshielded aromatic C—H group (FIG. 12(c)). The solid-state NMR methods were verified using compound c with a known crystal structure.

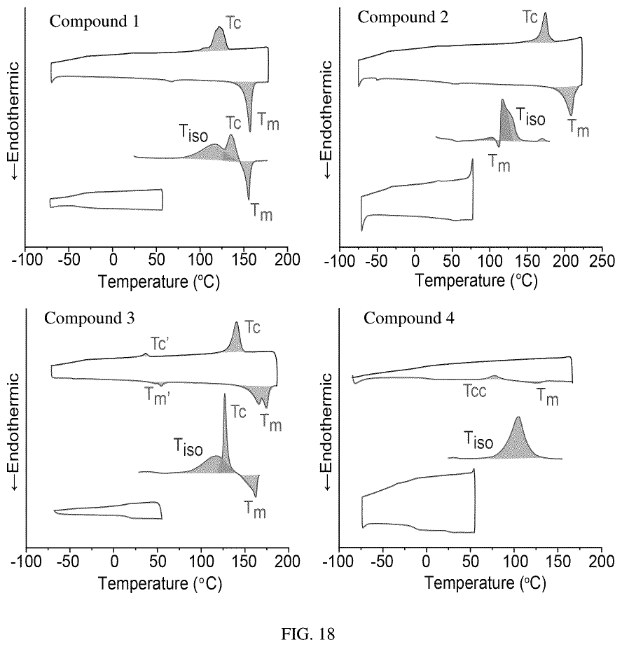

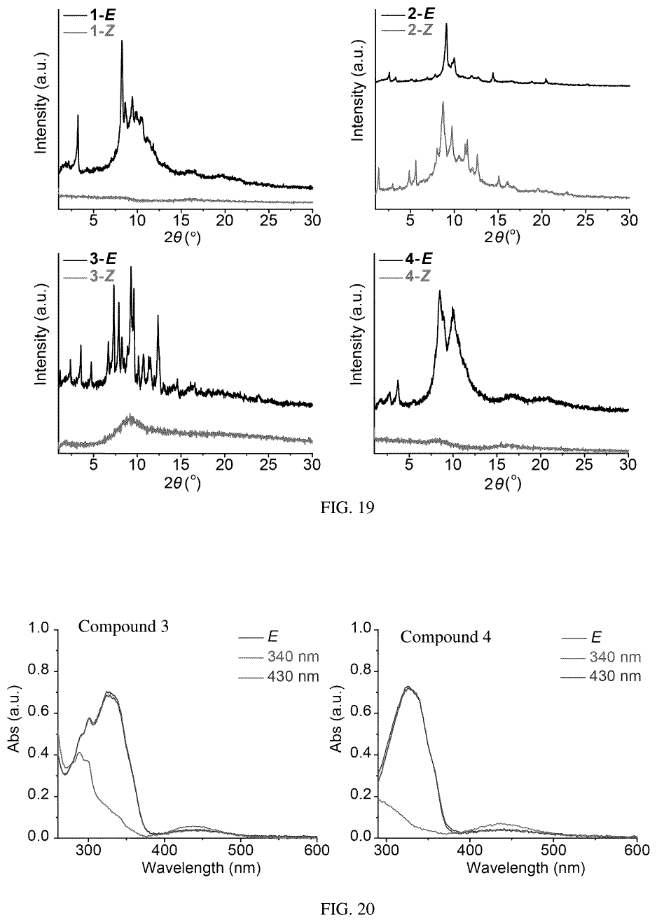

The new compounds 1, 3, and 4 exhibit a phase transition between E (crystalline) and Z (liquid), determined by differential scanning calorimetry (DSC). Compound 1-E shows a set of melting and crystallization peaks, whereas 1-Z remains a liquid over a wide range of temperatures (−35° C. to 60° C.) and an amorphous solid below its glass transition temperature at −35° C. (FIG. 2). Above 60° C., a thermally-triggered reverse isomerization (Z to E) occurs, and the crystalline 1-E is regenerated (FIG. 18). The relative phase of E and Z isomer is also confirmed by powder X-ray diffraction (FIG. 19), and the optical images corroborate the distinct phase of each isomer (FIG. 3). Compounds 3 and 4 show comparable thermal, diffraction, and phase properties as compound 1, as illustrated in FIGS. 19-20. Compound 2, on the other hand, exhibits crystallinity as both E and Z isomers; 2-Z melts at a lower temperature (110° C.) than 2-E (213° C.), indicating weaker intermolecular interactions among Z than E isomers (FIG. 18).

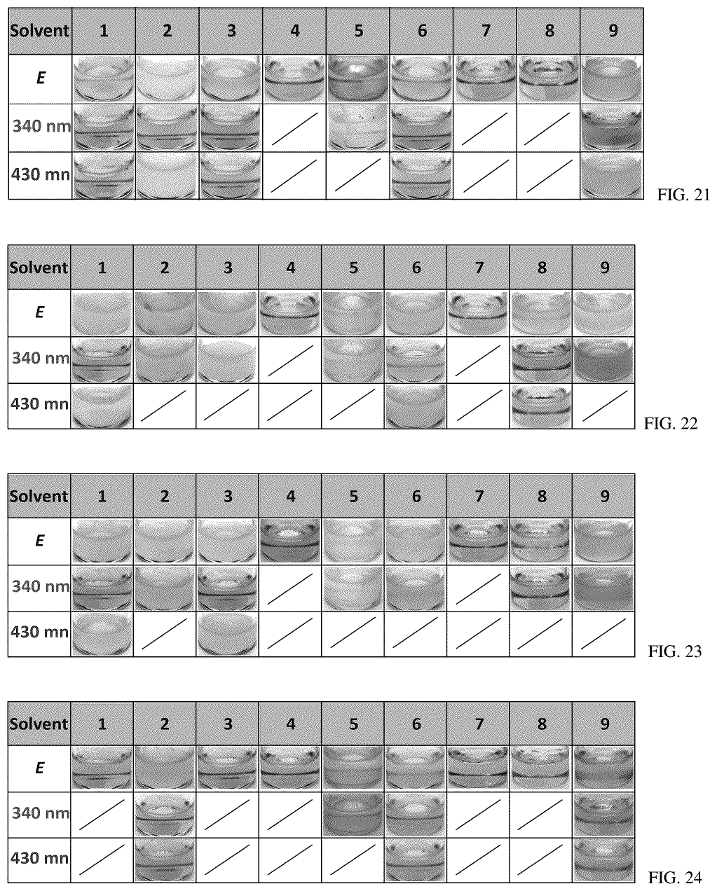

All compounds show reversible photoswitching between E and Z upon irradiation at 340 nm and 430 nm (FIG. 4, FIG. 5, and FIG. 20) in dilute solutions (−1.7×10⁻⁵ M). To find solvents in which the ratio between the maximum concentrations of Z and E isomers at the respective photostationary state (PSS) (i.e., r=[Z]max/[E]max) is the greatest, a solvent screening test for each compound was performed (FIGS. 21-24). As summarized in Table 1 below, the greatest r value of compound 1 is achieved in acetonitrile (r=10.4), that of compound 2 in methanol (r=8.8), compound 3 in acetone (r=5.7), and compound 4 in toluene (r=13.9). Despite its significant Z/E solubility change, Compound 4 was not suitable for recoverable catalysis due to the unfavorable precipitation of 4-E upon the photochemical triggering at 430 nm. This is attributed to the weak intermolecular interactions between 4-E, which was inferred from its lower melting point and melting enthalpy compared to those of other compounds (FIG. 18).

Thus, compounds 1 and 2 were selected as the most promising candidates for photoswitchable catalyst activation and recovery, based on their greater r values than that of compound 3.

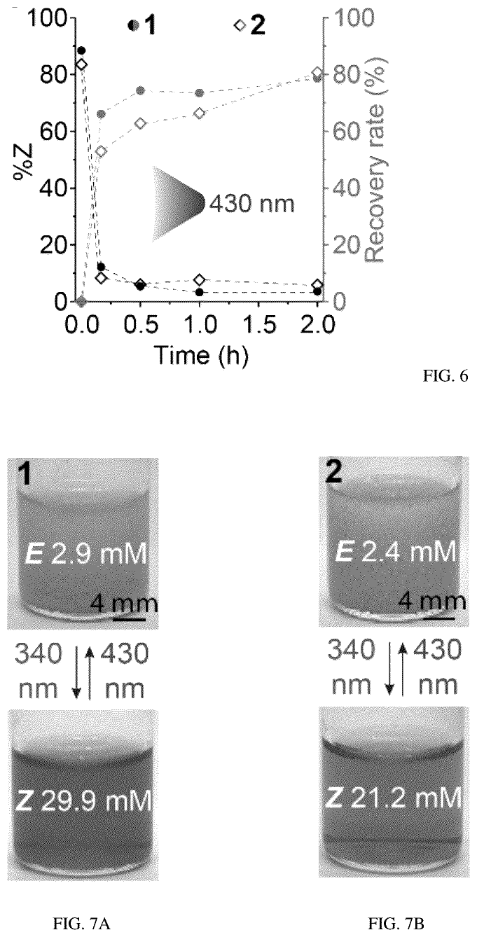

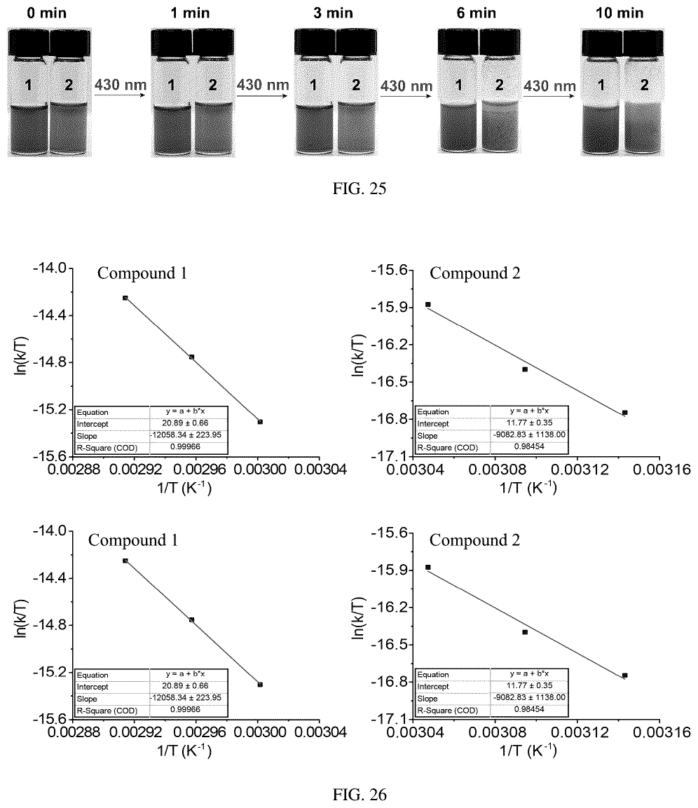

FIG. 6 and FIG. 25 illustrate the efficiencies of 430 nm induced catalyst precipitation and recovery for compounds 1 and 2, which were obtained by weighing the filtered precipitates after irradiation. Within 10 min of 430 nm irradiation, both compounds underwent Z-to-E isomerization, shown as the % Z drop of 75% in the suspension, and precipitation (>50%), which was followed by gradual additional precipitation that resulted in about 80% recovery within two hours. The full recovery of the catalysts is limited by the PSS ratios of compounds at 430 nm (82% for 1- E and 84% for 2-E). The intrinsic limitation stems from the suboptimal photoswitching property of pristine azobenzene (83% E at 430 nm), which could be overcome by the incorporation of other photoswitches that exhibit near-quantitative (about 100%) photoisomerization between E and Z, such as arylazopyrazole (>98%), azobispyrazole (>98%), hemithioindigo (>97%), or dithienylethene (˜95%).

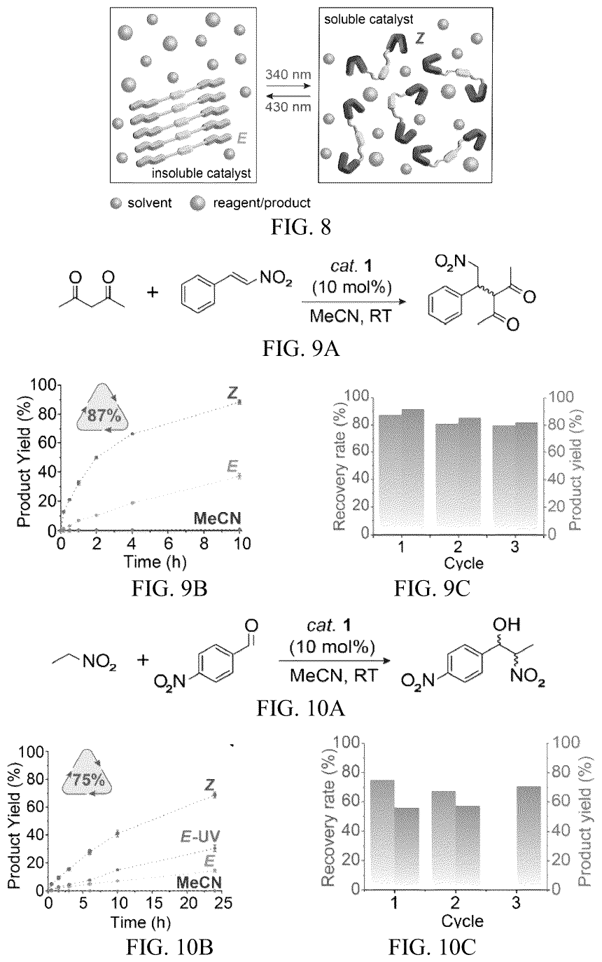

The reversible dissolution and precipitation of the compounds are clearly displayed as the changing opacity of solution under the alternating irradiation at 340 nm and 430 nm (FIG. 7A, FIG. 7B). Such photo-induced solubility changes of isomers are attributed to the significant configuration and polarity changes of azobenzene units in the catalyst structures. The increased steric hindrance of azobenzene moieties upon the E to Z isomerization effectively disrupts the self-assembly of linear catalysts, reducing the H-bonding and 71 interactions (FIG. 8). In acetonitrile and methanol, this effect is further enhanced by the increased polarity of the Z-azobenzene units.

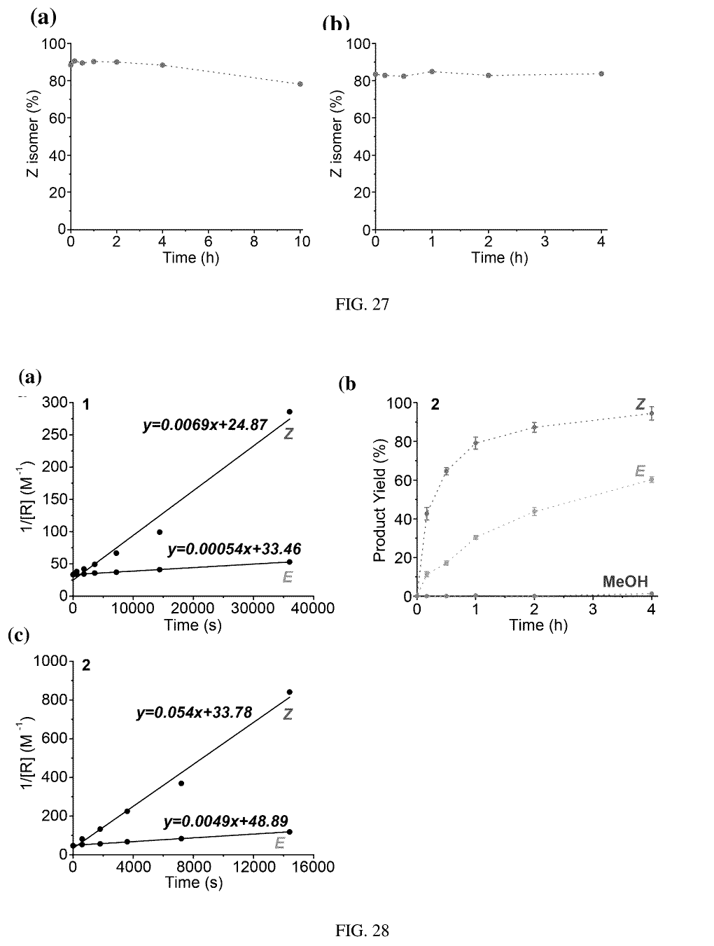

Having achieved photo-switchable catalysts that exhibit an about 10-fold solubility difference between E and Z isomers in select solvents, their catalytic activity was evaluated, as well as reusability in common base-catalyzed reactions. Michael addition, an effective synthetic method for diverse natural and biologically active compounds, was first selected to test the photo-induced activation and precipitation of catalysts, using the exemplary reaction shown in FIG. 9A. In the Michael addition, trans-β-nitrostyrene is a common reactant that exhibits high reactivity but hinders the selective photo-activation of azobenzene catalysts (compound 1-E 340 nm=24004 M⁻¹ cm⁻¹; compound 2-Eε340 nm=25623 M⁻¹ cm⁻¹) through its photo-isomerization and strong absorption at 340 nm (340 nm=11327 M⁻¹ cm⁻¹). Therefore, the catalysts were pre-activated in a separate solution (acetonitrile for 1; methanol for 2) and added to the reactants at room temperature. The spontaneous thermal reversion (Z to E) of 1-Z or 2-Z during the reaction is negligible due to the long half-lives at room temperature (8.3 days for 1-Z; 3.5 days for 2-Z; FIGS. 26-27 and Table 2 below), allowing the catalysts to remain dissolved during the reactions. Under reaction conditions, the Z-rich catalyst 1 showed a remarkably higher reaction rate (kz=6.9×10−3 M⁻¹s⁻¹) and product yield (2.4-fold) in 10 hours, compared to its E counterpart, kE=5.4×10⁻⁴ M⁻¹s⁻¹ (FIG. 7A, FIG. 28(a)). In the insoluble E isomeric state, the catalytic sites are less accessible to reagents, reducing the rate of catalysis. Upon E to Z isomerization, the number of available catalytic sites, or the concentration of catalysts in the reaction medium, markedly increases, which facilitates the desired catalysis reactions.

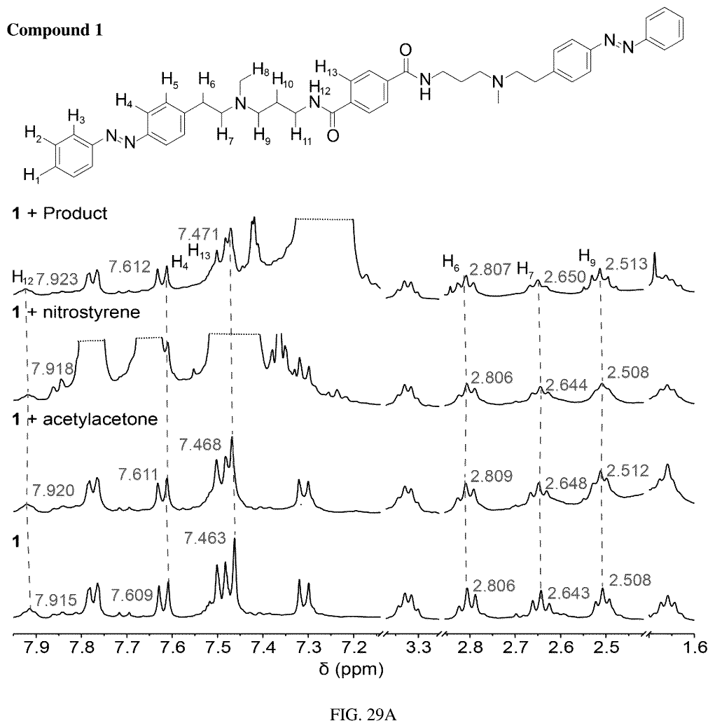

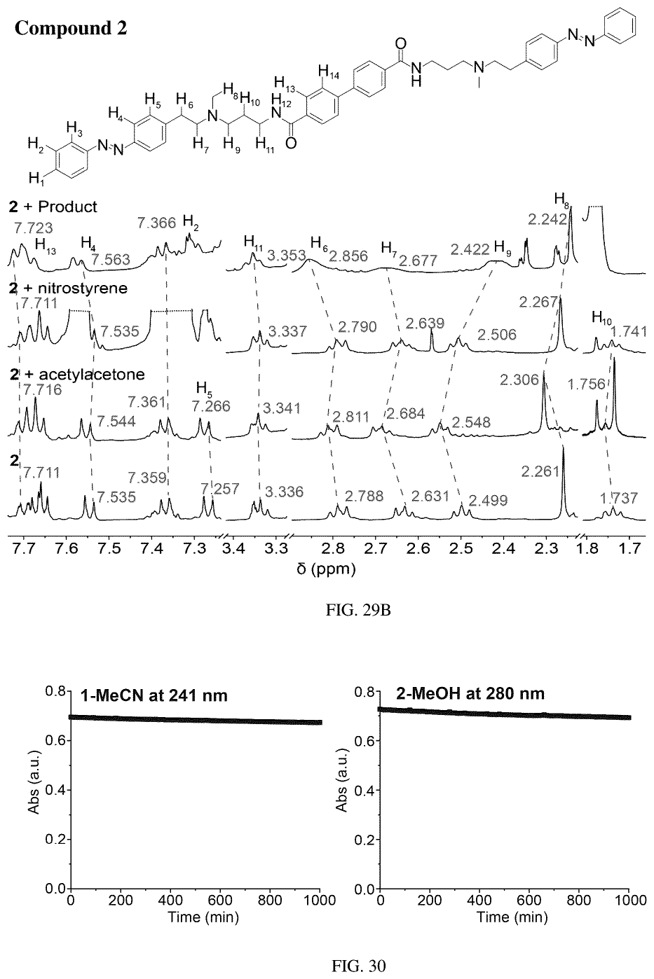

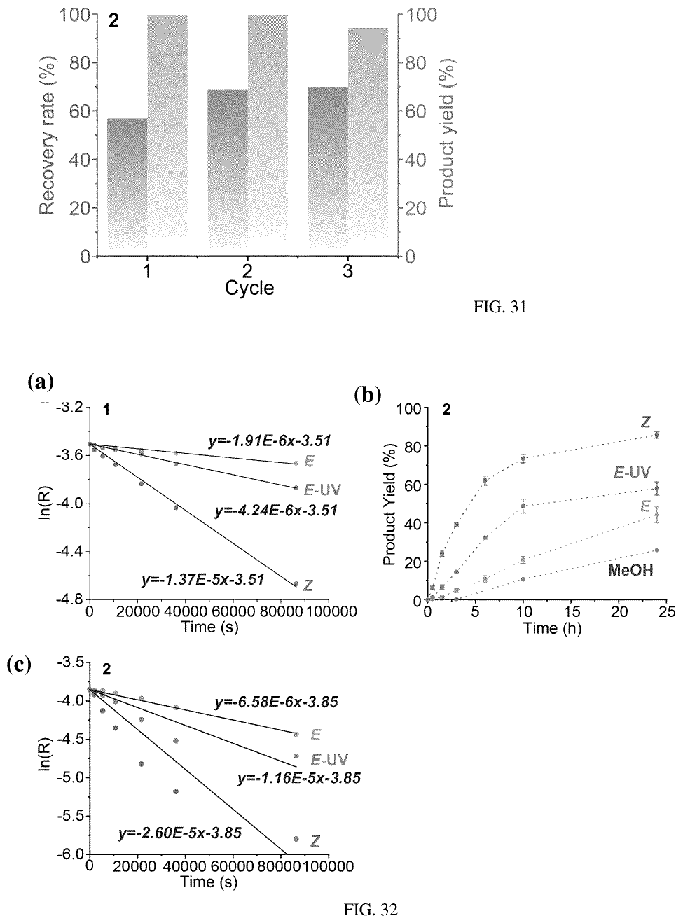

Upon completion of the reaction, the mixture was irradiated at 430 nm to trigger the Z→E isomerization (see Table 3 below), resulting in 87% catalyst recovery by filtration (FIG. 9C). The recovery rate of catalyst from the reaction mixture was higher than that measured from pristine acetonitrile (80%), indicating that the presence of product in the reaction medium does not hinder the self-assembly and precipitation of 1-E generated upon photoirradiation. To verify this, NMR analyses were performed where weak H-bonding and π-interactions were detected between the product and catalyst 1-E in the form of a small downfield chemical shift of amide N—H (Δδ=0.008) as well as aromatic and alkyl C—H (Δδ=0.001-0.008) (FIG. 29A). The recovered catalyst was reused for the subsequent two reactions, with identical 10 mol % loading, and the recovery rate of catalyst 1 as well as the product yield slightly decreased to 80% and 82%, respectively. Without being found by theory, the gradual loss of catalyst activity and recovery may have resulted due to one or more of the following factors: trace amounts of catalyst photo-degraded during repeated irradiations (FIG. 30); chemisorbed products or reactants to catalytic sites; or trace amounts of side products that poison catalysts. The activation and precipitation of catalyst 2 in methanol for the Michael addition are illustrated in FIG. 28(b), FIG. 28(c), and FIG. 31. Due to the activation of acetylacetone by methanol, the reaction proceeds rapidly and reaches completion in 4 hours. The catalyst recovery achieved upon 430 nm irradiation was only 57-70%, in contrast to 81% measured in pristine methanol. Stronger 7-interactions between the product and catalyst 2, shown as larger chemical shifts of aromatic and alkyl C—H (Δδ=−0.077−0.068) (FIG. 29B), provide a likely explanation for this reduction in catalyst recovery rate.

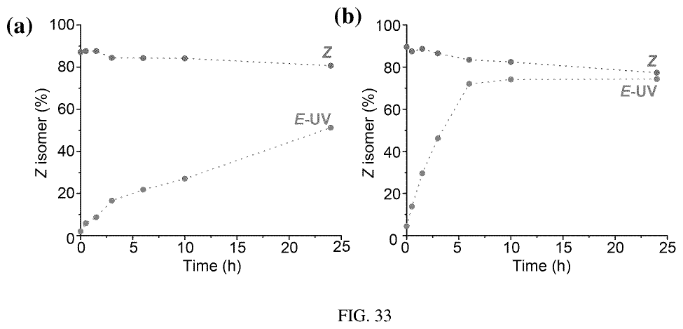

The photo-switchable base catalysts were further tested for the Henry (nitroaldol) reaction (FIG. 10A) that yields β-nitroalcohols (exemplary reaction shown in FIG. 7d), which can be easily transformed into useful components for organic synthesis. The reagents of the Henry reaction have a substantially lower absorption (4-nitrobenzaldehyde ε340 nm=323 M⁻¹ cm⁻¹) than catalysts (Table 4 below) at 340 nm, allowing the catalysts to be UV-activated in-situ (blue, FIG. 7e) or preactivated (FIG. 7e, red) to form soluble Z isomers that catalyze the reaction. Within 24 hours, the pre-activated 1-Z yields about 70% of product (kZ=1.37×10⁻⁵ s⁻¹), while the in-situ UV-activated catalyst shows slower reaction kinetics (kE-UV=4.24×10⁻⁶ s⁻¹) (FIG. 32(a)). The initial concentration of solubilized Z catalyst is very low in the in situ UV-activated system, which results in a low starting activity (similar to 1-E, orange, kE=1.91×10−6 s−1) and a gradual increase of the product yield over the prolonged irradiation. In contrast to the facile isomerization of compound 1 in pristine acetonitrile (˜88% Z at PSS), the in-situ UV-induced E→Z isomerization in the reaction medium is slower (48% Z after 24-hour irradiation) (FIG. 33(a)). Despite the low 340 nm of 4-nitrobenzaldehyde, its presence causes interference with the isomerization of the catalyst due to its high concentration (30 mM) in the reaction mixture.

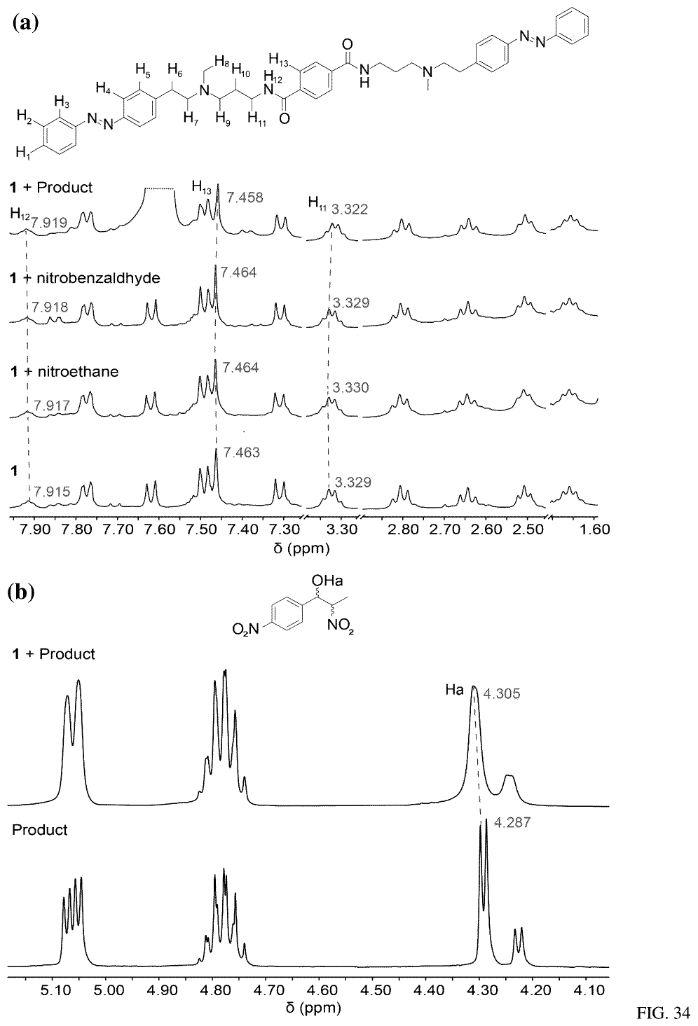

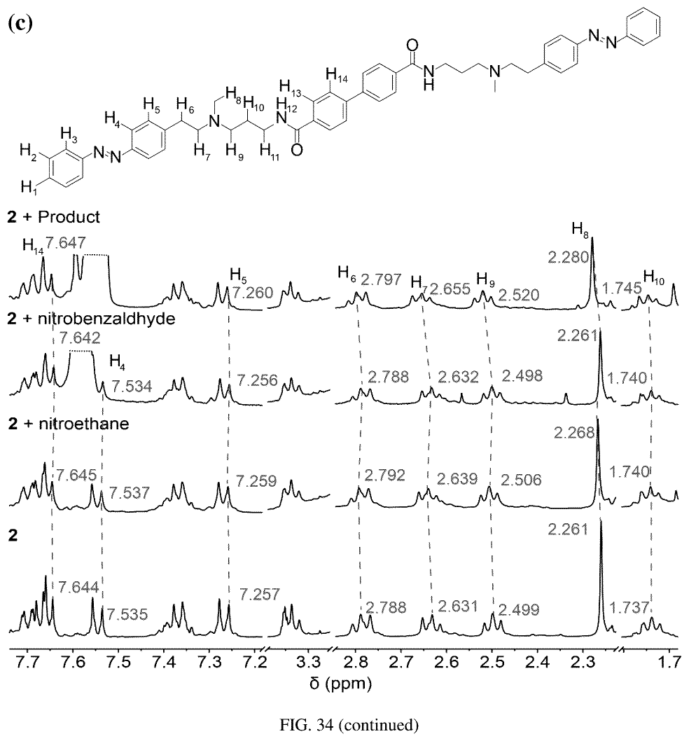

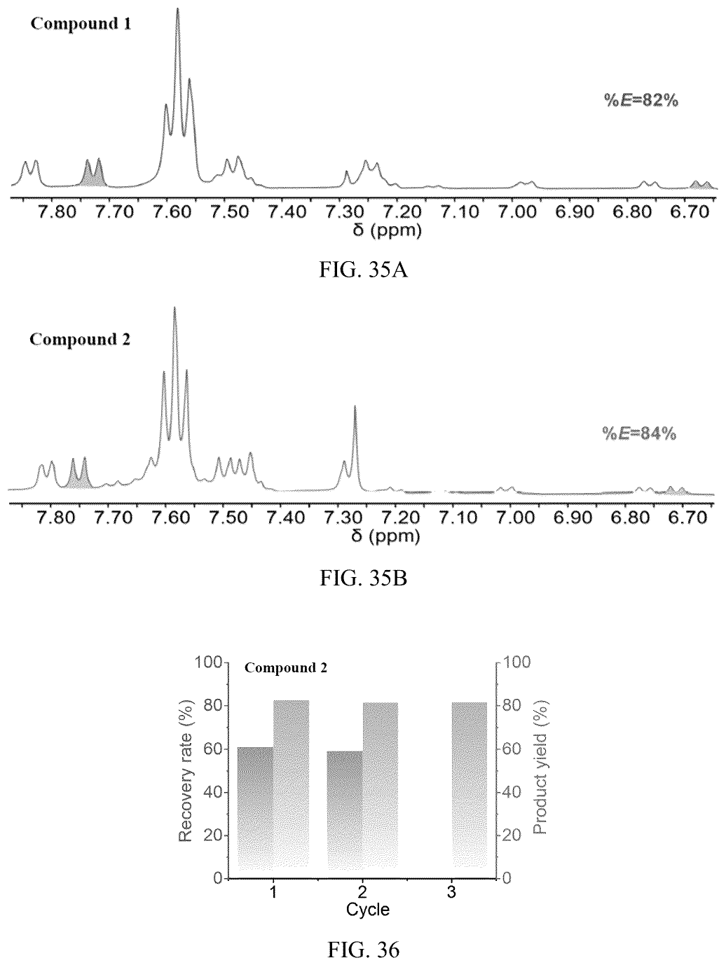

Following a 16-hour Henry reaction to assess the recovery of the catalyst, precipitation of the catalysts was induced by 430 nm irradiation, giving about 75% recovery, which is slightly less than the 80% recovery achieved in pristine acetonitrile. Without being bound by theory, the lower catalyst recovery can be attributed to the intrinsically suboptimal PSS of azobenzene, increased polarity of the mixture, and the noncovalent interactions between the catalyst and product (FIG. 24(a)). The H-bonding interaction between the hydroxy group of the product and catalyst 1 (10 mol %) yielded a significant chemical shift change (Δδ=0.018) (FIG. 34(b)), demonstrating the non-covalent interactions between the catalyst and the product. A longer reaction (24 hours) was performed for the third Henry reaction (FIG. 10C) to increase the yield of product (70%) compared to the 1st (56%) and 2nd (57%) reactions. Due to the higher concentration of product, precipitation of the catalyst was not achieved, despite successful Z to E photo-isomerization (FIG. 35A and FIG. 35B).

Pre-activated 2-Z afforded 85% product yield for the Henry reaction performed in methanol over 24 hours. The in-situ UV activation of catalyst 2 occurred rapidly in methanol, resulting in reaction kinetics (kE-UV=1.16×10 ⁻⁵ s⁻¹) similar to that of preactivated catalyst 2-Z (kZ=2.60×10⁻⁵ s⁻¹) (FIGS. 32(b)-32(c)). After a 16-hour reaction, the 430 nm induced precipitation of Z isomers led to about 60% recovery (FIG. 36), which was lower than the precipitation yield of 81% obtained in pristine methanol, providing similar results to catalyst 1. Upon completion of the third Henry reaction in both reactions with catalysts 1 and 2, solvent removal resulted in a liquid-phase mixture of the product, catalyst, and unreacted substrates, indicating significant intermolecular interactions between the catalyst and other components of the reaction. In contrast, any shorter Henry reactions or Michael additions allowed for the facile precipitation and separation of catalysts, highlighting the importance of selecting catalyst-product pairs that do not interfere with the catalyst self-assembly in order to achieve successful catalyst recovery and recycling. Ultimately, irreversible reactions with products that are removable from the reaction mixture via precipitation or evaporation will substantially enhance the recovery of photo-switchable catalysts.

In summary, the photo-responsive organocatalysts exhibit a significant solubility change when switched between their E and Z isomeric states. The photoswitchable amine catalysts demonstrated high recovery rates of 75-87% from the Michael addition and Henry reactions, as well as over 80% recovery after three rounds of Michael additions performed by the recycled catalysts. The viability of recoverable catalysts can be expanded through various compact catalytic motifs. The photoswitch units can then be selected or modified to separate the wavelengths that are used for photo-catalysis and photoswitching. The discovery of optically-controlled recoverable catalyst designs and reaction conditions that enable the facile recovery of catalysts, high yields of reactions, and successful recycling of catalysts are useful for the economical and sustainable catalysis in industrial settings.

In addition to the foregoing utilities described above, organic photoswitches that undergo reversible phase changes upon light irradiation have been integrated into various materials for applications including light-driven actuation, drug delivery, sensing, and optical memory (Han et al., “Optically-controlled Long-term Storage and Release of Thermal Energy in Phase-change Materials,” Nature Communications 8:1446 (2017), which is hereby incorporated by reference in its entirety). These additional utilities are also contemplated for the compounds and compositions described herein.

### Examples

The following examples are provided to illustrate this disclosure. The examples are merely illustrative and are not intended to limit compounds, compositions, methods, devices, and uses, materials, conditions, or process parameters as set forth therein.

### General Methods

All reactions were monitored by thin-layer chromatography (TLC) using Merck silica gel 60 F254 plates (0.25 mm). TLC plates were visualized using UV light (254 nm). Silica column chromatography was performed using Merck Silica Gel 60 (230-400 mesh). ¹H nuclear magnetic resonance spectography (¹H NMR and ¹³C NMR were recorded on a Varian INOVA 400 spectrometer at 400 MHz and 101 MHz. Solid-state ¹³C and ¹H-¹³C NMR spectra were recorded using a Bruker Neo Avance 400WB spectrometer at a ¹³C resonance frequency of 100 MHz. High-resolution mass spectra (ESI-MS) were recorded by the University of Illinois, Mass Spectrometry Lab. VWR hydrophobic PTFE filters (13 mm, 0.22 μm) were used for recovering precipitation in the cycling tests.

Ultraviolet-visible (UV-Vis) absorption spectra were obtained with a Cary 50 Bio UV-Vis spectrophotometer in a UV Quartz cuvette with a path length of 10 mm. Compounds were dissolved in solvents at a concentration of 0.0125 mg/ml unless otherwise specified. The UV-Vis absorption was first recorded in dark for 5 min, then samples were irradiated with a specified wavelength until no change in their absorbance was observed. Samples were irradiated with Thorlabs LEDs: M340L4 (340 nm, 2.22 μW/mm², 60 mW), M430L4 (430 nm, 35.3 μW/mm², 600 mW).

Differential scanning calorimetry (DSC) analysis was conducted on a DSC 250 (TA Instruments) with an RSC 90 cooling component. All samples were heated at a rate of 10° C./min unless otherwise noted. All E isomers were melted and cooled to −70° C. before reheating. Liquid Z isomers of compounds 1, 3, and 4 were first cooled to −70° C. then heated. Solid Z isomers of compound 2 were melted and cooled to −70° C. In DSC experiments with a scan rate of 20° C./min, the Z isomers were heated below their respective Tᵢₛₒ to prevent the Z-to-E thermal reversion. To determine the ΔHᵢₛₒ of Z isomers, samples were heated from 20° C. until the thermal isomerization was completed.

Z isomer samples were prepared for measurement by dissolving each E isomer in dichloromethane and irradiating the sample with 340 nm until a photostationary state was reached. Z-rich samples were concentrated, dried under vacuum, and then transferred to DSC pans for analysis.

X-ray powder diffraction (XRPD) in the 2θ range 0-30° (step size, 0.014°; time/step, 20 s; 0.04 rad Soller; 40 mA×60 kV) was collected on a Panalytical Empyrean diffractometer equipped with a GaliPIX3D line detector and in Bragg-Brentano geometry, using Mo-Kα radiation (λ=0.7093187° A) without a monochromator. Around 5 mg samples were loaded into capillary tubes (outer diameter=0.7 mm) and the measurements were carried out on the capillary spinner.

### Synthesis of Compounds

Scheme S1 shows the general synthetic route for intermediates S1, S2, and S3.

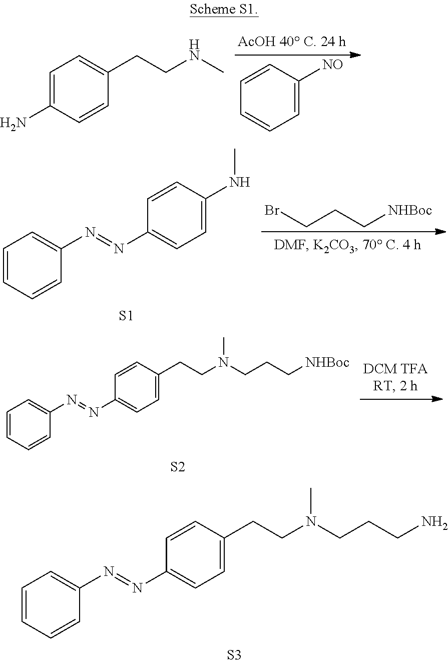

Synthesis of intermediate S1. To a solution of nitrosobenzene (313 milligram (mg), 2.93 millimole (mmol), 1 equivalent (eq) in glacial acetic acid (AcOH) 11 milliliters (mL)), 4-[2-(methylamino) ethyl]aniline (440 mg, 2.93 mmol, 1 eq)) was added. The reaction mixture was stirred at 40° C. for 24 hours (h). Then, the mixture was diluted with dichloromethane (DCM) (50 mL) and further neutralized by 5% NaHCO₃ aqueous solution. The two-layer mixture was extracted with DCM (50 mL) 3 times. The organic layer was collected, washed with brine, dried over anhydrous MgSO₄, and concentrated on a rotary evaporator. The crude product was further purified with silica gel chromatography (DCM:methanol (MeOH):triethylamine (Et₃N)=94:5:1) to yield an orange liquid S1 (450 mg, 64% yield); ¹H NMR (400 MHz, 298 K, CDCl₃), δ7.91-7.85 (m, 4H; Ar—H), 7.53-7.44 (m, 3H; Ar—H), 7.36-7.34 (d, 2H; Ar—H), 2.89 (s, 4H; CH₂), 2.46 (s, 3H; CH₃).¹³C NMR (101 MHz, 298 K, CDCl₃), δ152.81, 151.38, 143.73, 130.92, 129.54, 129.18, 123.14, 122.87, 53.13, 36.52, 36.29. HRMS (ESI): m/z calculated for C₁₅H₁₈N₃[M+H]⁺: 240.1422, found 240.1501.

Synthesis of intermediate S2. To a mixture of tert-butyl-3-bromopropylcarbamate (260 mg, 1.1 mmol, 1.3 eq) (t0-BOC) and S1 (200 mg, 0.84 mmol, 1 eq) in dry dimethyl formamide (DMF) (6 mL) was added potassium carbonate (260 mg, 1.9 mmol, 2.3 eq). The resulting mixture was heated at 70° C. for 4 h under N₂ atmosphere. Then, the solvent was removed under reduced pressure, and water (20 mL) was added to the residue. The crude product was extracted with DCM (30 mL) 3 times. The organic layer was collected, washed with brine, dried over anhydrous MgSO₄, and concentrated on a rotary evaporator. The crude product was further purified with silica gel chromatography (EtOAC:Et₃N=100:3) to yield an orange liquid S2 (199 mg, 60% yield); ¹H NMR (400 MHz, 298 K, CDCl₃), δ7.91-7.85 (m, 4H; Ar—H), 7.52-7.42 (m, 3H; Ar—H), 7.34-7.32 (d, 2H; Ar—H), 5.24 (b, 1H; NH) 3.17-3.15 (m, 2H; CH₂), 2.87-2.83 (t, 2H; CH₂), 2.67-2.63 (t, 2H; CH₂), 2.48-2.45 (t, 2H; CH₂), 2.29 (s, 3H; NCH₃), 1.68-1.59 (m, 2H; CH₂), 1.44 (s, 9H; CH₃). ¹³C NMR (101 MHz, 298 K, CDCl₃), δ156.13, 152.74, 151.18, 143.96, 130.84, 129.43, 129.10, 123.05, 122.81, 78.88, 59.29, 55.94, 42.03, 39.69, 33.73, 28.53, 27.05. HRMS(ESI): m/z calculated for C₂3H₃₃N₄O₂ [M+H]⁺: 397.2525, found 397.2599.

Synthesis of intermediate S3. S2 (140 mg, 0.35 mmol) was dissolved in DCM (0.5 mL) and trifluoracetic acid (TFA, 0.5 mL). The resulting mixture was stirred at room temperature (RT) for 1 hour (h). Then, the mixture was neutralized with 5% NaHCO₃ aqueous solution, and extracted with EtOAc (20 mL) 3 times. The organic layer was collected, washed with brine, dried over anhydrous MgSO₄, and concentrated on a rotary evaporator. The crude product was further purified with silica gel chromatography (DCM:MeOH:Et₃N=90:9:1) to yield an orange solid S3 (60 mg, 57% yield); ¹H NMR (400 MHz, 298 K, CDCl₃), δ7.91-7.84 (m, 4H; Ar—H), 7.54-7.44 (m, 3H; Ar—H), 7.36-7.34 (d, 2H; Ar—H), 4.99 (b, 2H; NH₂) 2.88-2.84 (t, 2H; CH₂), 2.72 (m, 2H; CH₂), 2.68-2.64 (m, 2H; CH₂), 2.49-2.46 (t, 2H; CH₂), 2.32 (s, 3H; NCH₃), 1.67-1.60 (m, 2H; CH₂). ¹³C NMR (101 MHz, 298 K, CDCl₃), δ152.76, 151.17, 144.19, 130.79, 129.44, 129.08, 122.97, 122.79, 59.37, 55.51, 42.17, 40.60, 33.73, 30.76. HRMS(ESI): m/z calculated for C₁₈H₂₅N₄[M+H]⁺: 297.2001, found 297.2065.

Scheme S2 shows a synthetic route for compound 1.

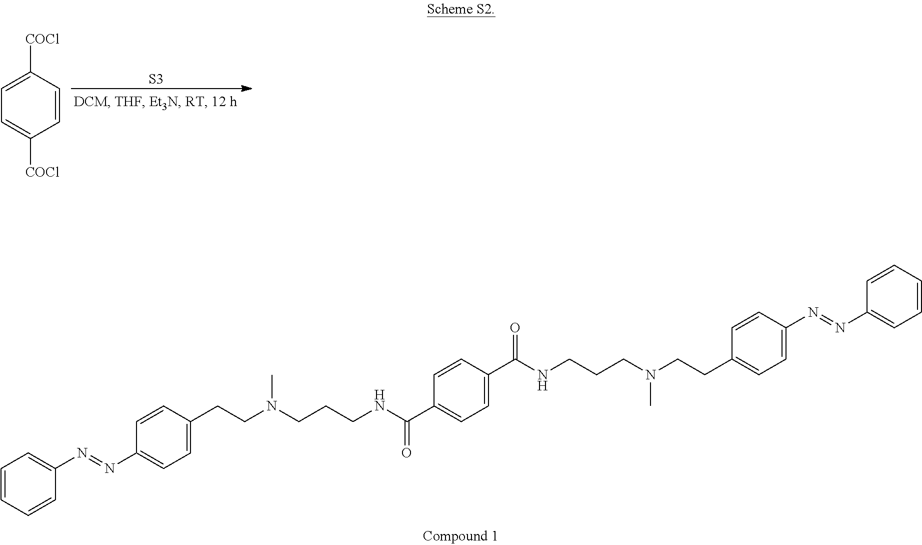 [SMILES: O=C(Cl)c1ccc(C(=O)Cl)cc1 ; "S3" ; CN(CCCNC(=O)c1ccc(C(=O)NCCCN(C)CCc2ccc(/N=N/c3ccccc3)cc2)cc1)CCc1ccc(/N=N/c2ccccc2)cc1]

Synthesis of compound 1. The terephthaloyl chloride (21.9 mg, 0.11 mmol, 1 eq) was dissolved in dry tetrahydrofuran (THF), 0.5 mL) which was added dropwise to the mixture of S3 (95.9 mg, 0.32 mmol, 2.9 eq) and Et₃N (0.2 mL, 1.4 mmol) in DCM (5 mL). After the gas evolution stopped, the mixture was stirred for 12 h under N₂ atmosphere. Then, the solvent was removed on a rotary evaporator. The obtained solid was washed with 5% NaHCO₃ aqueous solution (20 mL) and extracted with DCM (20 mL) 3 times. The organic layer was collected, washed with brine, dried over anhydrous MgSO₄, and concentrated on a rotary evaporator. The crude product was purified with silica gel chromatography (DCM:MeOH:Et₃N=90:9:1) to yield an orange solid (37 mg, 93% yield); ¹H NMR (400 MHz, 298 K, CD₂Cl₂), δ7.98 (s, 2H; NH), 7.79-7.77 (d, 4H; Ar—H) 7.67-7.65 (d, 4H; Ar—H), 7.53 (s, 4H; Ar—H), 7.45-7.37 (m, 6H; Ar—H), 7.22-7.20 (d, 4H; Ar—H), 3.43-3.38 (q, 4H; CH₂), 2.79-2.76 (t, 4H; CH₂), 2.68-2.62 (t, 4H; CH₂), 2.56-2.53 (t, 4H; CH₂), 2.25 (s, 6H; NCH₃), 1.71-1.65 (m, 4H; CH₂). ¹³C NMR (101 MHz, 298 K, CDCl₃), δ166.29, 152.69, 151.19, 143.37, 137.10, 130.89, 129.27, 129.11, 126.98, 123.11, 122.84, 59.54, 57.21, 41.96, 40.61, 33.54, 25.29. HRMS(ESI): m/z calculated for C₄₄H₅₁N₈O₂ [M+H]⁺: 723.4135, found 723.4130.

Scheme S3 shows the synthetic route for compound 2.

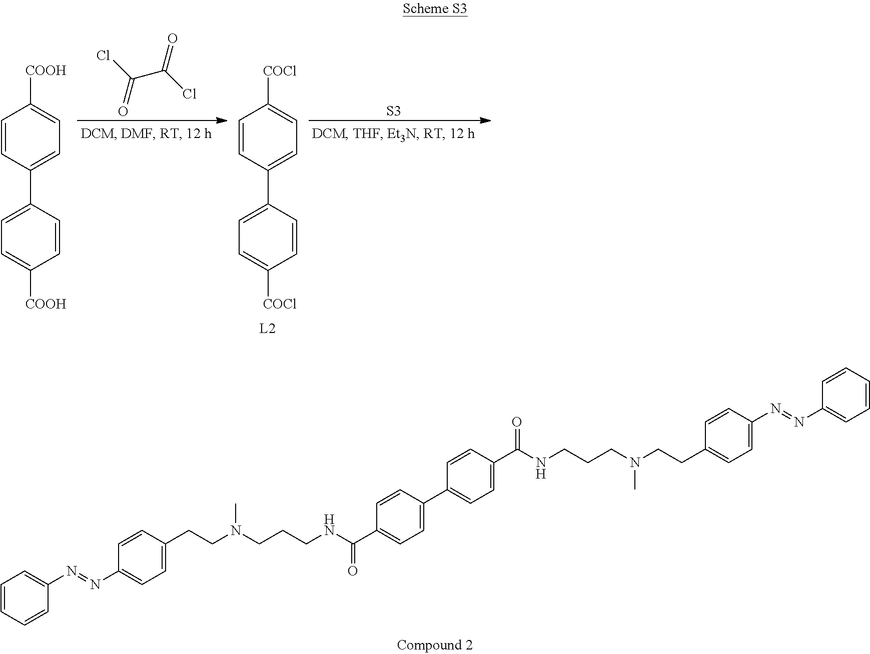 [SMILES: O=C(O)c1ccc(-c2ccc(C(=O)O)cc2)cc1 ; O=C(Cl)C(=O)Cl ; O=C(Cl)c1ccc(-c2ccc(C(=O)Cl)cc2)cc1 ; "S3" ; CN(CCCNC(=O)c1ccc(-c2ccc(C(=O)NCCCN(C)CCc3ccc(/N=N/c4ccccc4)cc3)cc2)cc1)CCc1ccc(/N=N/c2ccccc2)cc1]

Synthesis of L2 and compound 2. To a solution of [1,1′-biphenyl]˜4,4′-dicarboxylic acid (40 mg, 0.16 mmol, 1 eq) in DCM (3 mL), oxalyl chloride (36 μL, 0.42 mmol, 2.6 eq) was added dropwise at room temperature and stirred for 10 min. A catalytic amount (a drop) of DMF was added to the mixture, and the solution was stirred overnight under N₂ atmosphere, generating CO₂ (g), CO (g), and HCl (g) through a bubbler. The reaction mixture was dried under vacuum for 2 h to obtain L² (light brown solid) which was used for the next step without further purification. Compound 2 was prepared by following the same procedure of compound 1. The crude product was purified with silica gel chromatography (DCM:MeOH:Et₃N=94:5:1) to yield an orange solid (32 mg, 50% yield); ¹H NMR (400 MHz, 298 K, CD₂Cl₂), δ8.01 (s, 2H; NH), 7.82-7.80 (d, 4H; Ar—H) 7.77-7.75 (d, 4H; Ar—H), 7.66-7.64 (d, 4H; Ar—H), 7.55-7.53 (d, 4H; Ar—H), 7.51-7.44 (m, 6H; Ar—H), 7.34-7.32 (d, 4H; Ar—H), 3.56-3.51 (q, 4H; CH₂), 2.92-2.88 (t, 4H; CH₂), 2.78-2.75 (t, 4H; CH₂), 2.66-2.63 (t, 4H; CH₂), 2.37 (s, 6H; NCH₃), 1.82-1.76 (m, 4H; CH₂). ¹³C NMR (101 MHz, 298 K, CDCl₃), δ166.70, 152.66, 151.23, 143.50, 142.72, 134.00, 130.92, 129.32, 129.12, 127.45, 127.16, 123.14, 122.84, 59.66, 57.30, 42.10, 40.60, 33.59, 25.45. HRMS(ESI): m/z calculated for C₅₀H₅₅N₈O₂[M+H]⁺: 799.4448, found 799.4445.

Scheme S4 shows a synthetic route for compound 3.

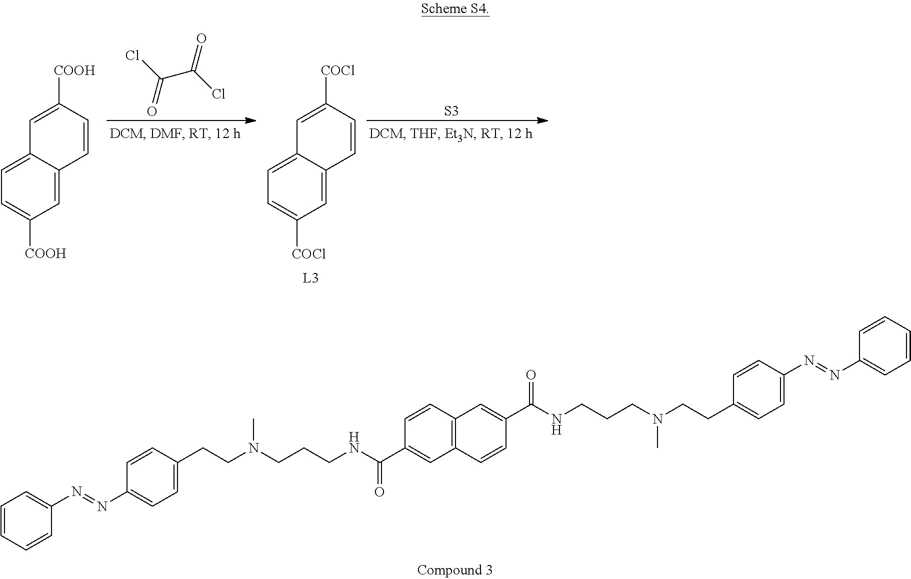 [SMILES: O=C(O)c1ccc2cc(C(=O)O)ccc2c1 ; O=C(Cl)C(=O)Cl ; O=C(Cl)c1ccc2cc(C(=O)Cl)ccc2c1 ; "S3" ; CN(CCCNC(=O)c1ccc2cc(C(=O)NCCCN(C)CCc3ccc(/N=N/c4ccccc4)cc3)ccc2c1)CCc1ccc(/N=N/c2ccccc2)cc1]

Synthesis of L3 and compound 3 followed the same procedure as for compounds L2 and compound 1, using 2,6-naphthalenedicarboxylic acid (40 mg, 0.18 mmol) as starting material. The crude product was collected by filtration and washed with cold DCM. The solid was recrystallized in CH₃CN to yield an orange solid (31 mg, 44% yield);¹H NMR (400 MHz, 298 K, CD₂Cl₂), δ8.22-8.20 (t, 2H; Ar—H), 8.12 (s, 2H; NH), 7.82-7.78 (m, 6H; Ar—H), 7.71-7.69 (d, 2H; Ar—H), 7.67-7.65 (d, 4H; Ar—H), 7.52-7.43 (m, 6H; Ar—H), 7.29-7.27 (d, 4H; Ar—H), 3.59-3.55 (q, 4H; CH₂), 2.93-2.90 (t, 4H; CH₂), 2.82-2.78 (t, 4H; CH₂), 2.71-2.68 (t, 4H; CH₂), 2.42 (s, 6H; NCH₃), 1.87-1.81 (m, 4H; CH₂). ¹³C NMR (101 MHz, 298 K, CDCl₃), δ166.84, 152.67, 151.17, 143.47, 133.90, 133.53, 130.90, 129.24, 129.11, 128.87, 127.07, 124.36, 123.13, 122.88, 59.71, 57.44, 42.24, 40.86, 33.69, 25.45. HRMS(ESI): m/z calculated for C₄₅H₅₃N₈O₂[M+H]⁺: 773.4291, found 773.4285.

Scheme S5 shows a synthetic route for compound 4.

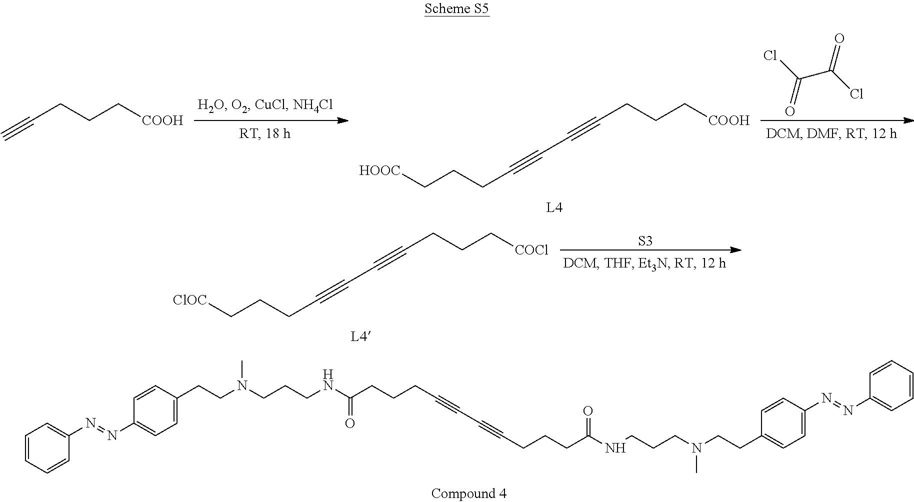

Synthesis of L4, L4′ and compound 4. L4 was prepared according to the previous literature procedures. (C. Gagnon, et al., Science 2020, Vol. 367, 917-921.) L⁴′ and compound 4 were prepared by following the same procedure of L² and compound 1, respectively, using L⁴ (31 mg, 0.14 mmol) as starting material. The crude product was further purified with silica gel chromatography (DCM:Et₃N=10:1) to yield an orange solid (24 mg, 44% yield); ¹H NMR (400 MHz, 298 K, CDCl₃), δ7.91-7.85 (dd, 8H; Ar—H), 7.53-7.44 (m, 6H; Ar—H), 7.36-7.34 (d, 4H; Ar-H), 6.69 (b, 2H; N—H) 3.30-3.26 (q, 4H; CH₂), 2.90-2.86 (t, 4H; CH₂), 2.73-2.69 (t, 4H; CH₂), 2.53-2.51 (t, 4H; CH₂), 2.31 (s, 6H; NCH₃), 2.26-2.22 (t, 4H; CH₂), 2.11-2.08 (t, 4H; CH₂), 1.78-1.73 (m, 4H; CH₂), 1.67-1.63 (m, 4H; CH₂). ¹³C NMR (101 MHz, 298 K, CD₃0D), δ175.63, 153.86, 152.87, 142.04, 132.28, 130.79, 130.26, 124.24, 123.76, 77.43, 66.98, 58.52, 55.33, 40.91, 37.45, 35.65, 31.89, 26.09, 25.53, 19.29. HRMS(ESI): m/z calculated for C₄₈H₅₉N₈O₂[M+H]⁺: 779.4761, found 779.4752.

### Solid-State NMR.

Experimental. Samples were packed into 4-mm zirconia rotors with KelF caps for magic-angle spinning (MAS) in a Bruker double-resonance probe head. ¹³C NMR spectra were recorded with multiple cross polarization (multiCP) (R. L. Johnson, K. Schmidt-Rohr, J. Magn. Reson. 2014, 239, 44-49) at 14 kHz MAS, with recycle delays of 6 to 10 s and four repolarization delays of 3 to 5 s between cross polarization periods of 1.1 ms duration. Composite ¹³C pulse (P. Duan, K. Schmidt-Rohr, J. Magn. Reson. 2017, 285, 68-78) and a linear amplitude ramp from 85 to 100% for ¹H were employed. Detection was started after two rotation periods with a centered, phase-cycled ¹³C 180° pulse that generates a Hahn spin echo, avoiding spectral baseline distortions. Pulsed high-power ¹H decoupling with |γB₁|/2π=86 kHz was applied during a detection period of 18 ms duration. In addition, the same experiment was performed with recoupled dipolar dephasing (J. D. Mao, K. Schmidt-Rohr, Environ. Sci. Technol. 2004, 38, 2680-2684) of 0.068 ms duration centered on the ¹³C 180° pulse was used to obtain selective spectra of carbons not bonded to hydrogen and mobile segments. Chemical shifts relative to TMS were externally referenced by the ¹³COO carbon of the α-modification of glycine at 176.49 ppm.

Signals of CHₙ segments undergoing motions with rates near the ¹³C Larmor frequency (2π×100 MHz) were identified by multiCP followed by ¹³C spin-lattice relaxation during a 2-s period with the magnetization stored along ±z (D. A. Torchia, J. Magn. Reson. 1978, 30, 613-616). The difference relative to the unrelaxed spectrum is shown. It resembles the direct-polarization spectrum with a 2-s recycle delay but is free of intensity distortions by heteronuclear nuclear Overhauser enhancement. In addition, the extent of relaxation can be assessed more easily.

Two-dimensional ¹H-¹³C NMR heteronuclear correlation (HetCor) spectra were recorded at 7.5 kHz MAS. Homonuclear decoupling during ¹H evolution was achieved by frequency-switched Lee-Goldburg (A. Bielecki, A. C. Kolbert, H. J. M. De Groot, R. G. Griffin, M. H. Levitt, Adv. Magn. Opt. Reson. 1990, 14, 111-124) off-resonance irradiation at |γB₁|/2 π=95 kHz. After cross polarization for typically 0.4 ms, transferring magnetization over a few bond distances, and total sideband suppression (TOSS) (W. T. Dixon, J. Chem. Phys. 1982, 77, 1800-1809) by four ¹³C 180° pulses, the ¹³C magnetization was detected with high-power ¹H decoupling. For probing alkyl-aromatic proximity on the 0.5 nm scale, a longer CP time of 1.1 ms was used.

Quantum-chemical calculations of ¹³C chemical shifts were performed using Gaussian 16 (M. J. Frisch, et al. Gaussian 16, 2016, Rev. B. 02, Wallingford,CT). Different molecular conformations were generated by setting and freezing up to two related torsion angles, and then optimized at the M062X/6-31+G(d,p) level of theory. NMR magnetic shielding values of these relaxed geometries were calculated using the Gauge Including Atomic Orbitals (GIAO) method (R. Dltchfield, J. Chem. Phys. 1972, 56, 5688-5691) at the mPW1PW91/6-311+G(2d,p) level of theory, and then converted to ¹³C chemical shifts using scaling factors published by M. W. Lodewyk, et al., Chem. Rev. 2012, 112, 1839-1862).

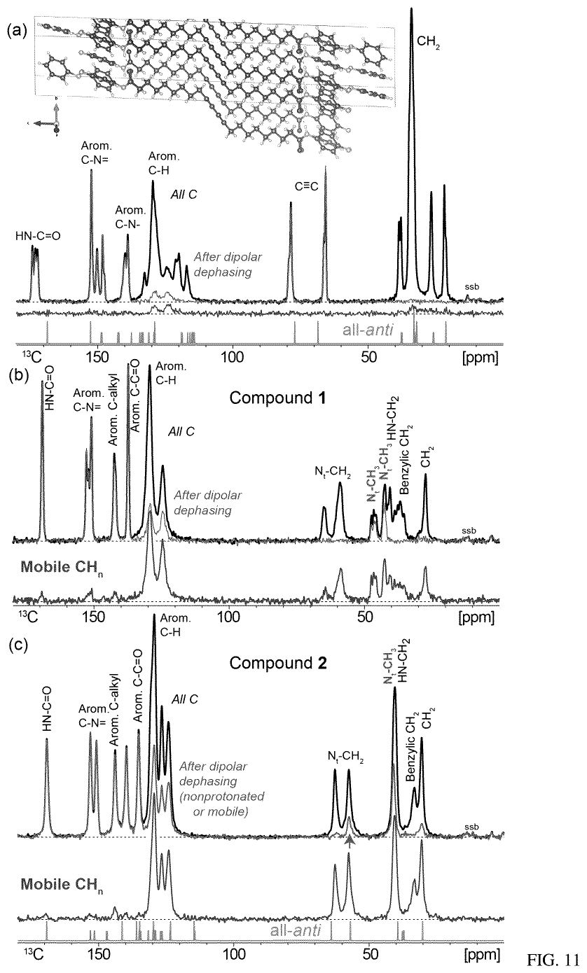

Solid-state NMR characterization of molecular packing and dynamic conformation (FIG. 11). The molecular packing and dynamic conformation of compound 2 was characterized by solid-state ¹H-¹³C NMR, where high amide ¹H chemical shifts of about 9 ppm are indicative of H-bonding while low aromatic chemical shifts ≤6 ppm reflect proximity of aromatic H to the center of a neighboring aromatic ring, for instance in a herring-bone packing of the rings due to aromatic CH—π interactions. Comparison of experimental ¹³C chemical shifts, see FIG. 11, with quantum-chemical results for various conformations of the alkyl segments, combined with mobility information from NMR relaxation, gives insights into their conformation and relative packing density. Long-range alkyl-aromatic cross peaks in ¹H-¹³C HetCor NMR with long cross polarization can be used to distinguish neat alternating aromatic and alkyl layers from more complex, mixed structures.

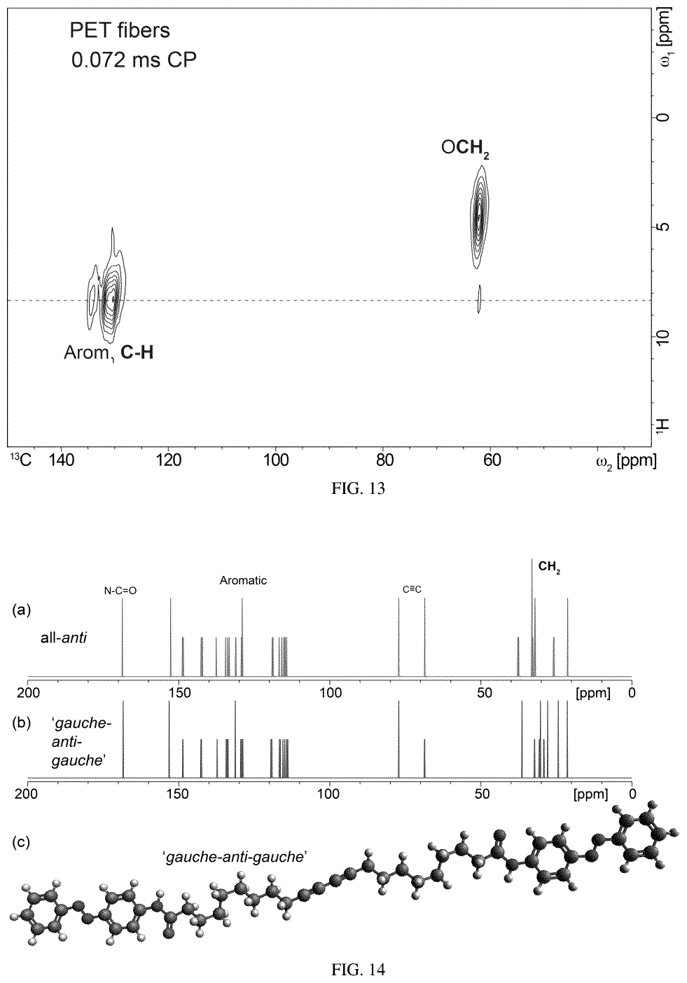

Solid-state NMR of reference materials. To start, NMIR reference data were obtained for the comparative diazobenzene diacetylene compound c (G. D. Han, S. S. Park, Y. Liu, D. Zhitomirsky, E. Cho, M. DincǍ, J. C. Grossman, J. Mater. Chem. A 2016, 4, 16157-16165) with a known single-crystal X-ray structure, as shown in FIG. 11(a). This structure documents H-bonding through stacked amide groups; accordingly in NMR, the benzylic amide protons in FIG. 12(a) exhibit high chemical shifts near 10 ppm. For reference, in another benzylic amide, N—H . . . N hydrogen bonding produced an amide ¹H chemical shift of only 8.1 ppm. The azobenzene aromatic rings of neighboring molecules in the X-ray structure interact through herring-bone rather than parallel stacking. As a consequence, aromatic chemical shifts in FIG. 11(a) are reduced from ≥7.2 ppm in solution to ≤6.7 ppm; the proton of an azobenzene phenylene ring exhibits a chemical shift as low as 5.4 ppm. The large (about 3.3 ppm) difference between aromatic and N—H proton chemical shifts in FIG. 12(a), comparable to the aromatic to CH₂ difference, is noteworthy and rules out significant chemical-shift miscalibration. Similar spectral features indicative of aromatic CH—π interaction have been reported for poly(p-phenylene terephthalamide) (PPTA) fibers with their herring-bone packing of aromatic rings and hydrogen bonding of benzylic amides. By contrast, in poly(ethylene terephthalate) (PET) fibers with their parallel ring packing in the crystallites, observed high chemical shifts, >8.2 ppm, were observed, as in FIG. 13, close to predicted solution-NMR values. Incomplete dipolar dephasing and relatively fast spin-lattice relaxation of a moderate fraction of the aromatic C—H signals documents fast large-amplitude motions of the terminal phenyl rings, while the full dipolar dephasing (thin red trace in FIG. 11(a)) and slow spin-lattice relaxation (bottom green trace) of the CH₂ signals indicates immobilization of the octamethylene segments.

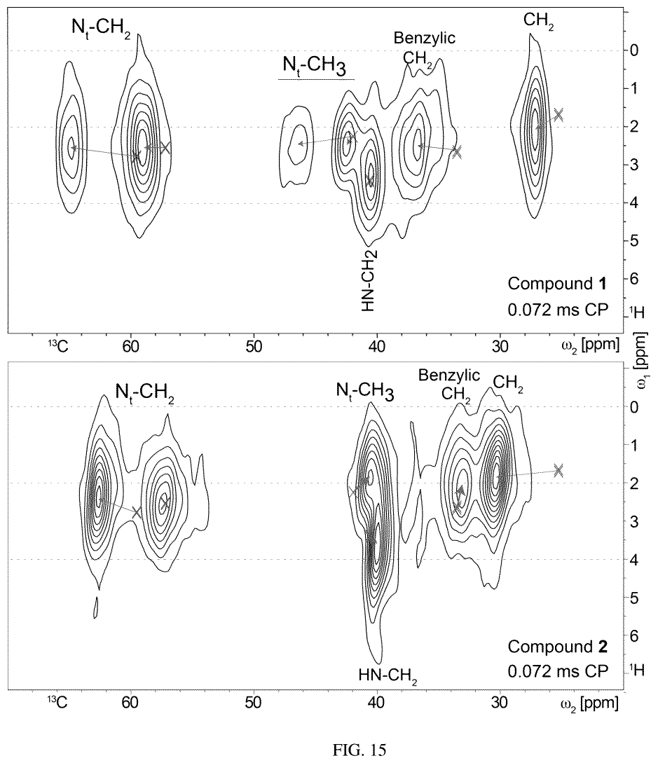

The absence of distinctive alkyl-proton cross peaks at the azobenzene C—N=carbons resonating near 150 ppm in FIG. 12(a) and FIG. 15(a) conforms with the segregation of aromatic and alkyl segments in the X-ray structure. The all-anti conformation of the octamethylene alkyl segments is confirmed by the good agreement of observed CH₂ chemical shifts with quantum-chemical calculations for this conformation (see stick spectrum at the bottom of FIG. 11(a). For reference, FIG. 14 shows a different plausible conformation and its significantly different predicted CH₂ spectral pattern.

¹³C chemical shifts and conformation of compounds 1 and 2. The corresponding NMR analyses were applied to assess the molecular packing and dynamic conformations of compound 1 and 2. Their spectra are shown in FIGS. 11(b), 11(c), 12(b), 12(c), 15(a), 15(b), 16(b), and 16(c). In FIG. 11(b), the ¹³C chemical shifts predicted through quantum chemical calculations for the all-anti conformation of the alkyl segment are in adequate agreement with experiment for compound 2, while the spectrum of compound 1 in FIG. 11(c) exhibits alkyl multiplets indicating conformational complexity and multiple molecules in the asymmetric unit cell.

Alkyl dynamic disorder in compound 2. Due to rotational jumps of the CH₃ group and fast phenylene ring flips, the ¹³C NMR signals near 44 and 128 ppm, respectively, of 1 and 2 undergo incomplete dipolar dephasing under gated ¹H decoupling (red-trace spectra). In strong contrast with the expected complete CH₂ dipolar dephasing in 1 and the diacetylene reference, compound 2 exhibits incomplete dipolar dephasing of the peaks of the Nₜ—CH₂—CH₂ groups (thin red line spectrum in FIG. 11b). This indicates a strong reduction in ¹H-¹³C dipolar couplings that is a hallmark of fast CH₂ motions of significant amplitude, which in turn indicates loose packing of the alkyl segments. One can conclude that the controlling intermolecular packing forces must be sought elsewhere in the structure of compound 2. The observed dynamic disorder in the alkyl segments of the crystal reduces the difference in molar entropy relative to the liquid, A_{fus}S, which according to Tₘ=Δ_{fus}H/Δ_{fus}S increases the melting point of 2.

¹H chemical shifts and intermolecular interactions in compound 1 and compound 2. In FIGS. 12(b) and 12(c), the N—H proton chemical shift detected at the amide C═O shows a +1-ppm increase, from 8.0 ppm in solution to 9 ppm in solid state, indicating hydrogen bonding in both 1 and 2. Whereas in solution the N—H chemical shift is within ˜0.4 ppm from the aromatic ¹H peaks, in the crystal the packing effects result in a larger ˜2.5 ppm separation between NH and aromatic ¹H (see FIG. 12(c)).

The aromatic and alkyl ¹H chemical shifts of compound 1 in the spectra of FIGS. 12(b) and 15(a) are fairly similar to the corresponding values in solution, indicating no pronounced CH—π interactions. The observed small upfield shifts of the aromatic ¹H might be attributed to lateral staggering of parallel aromatic rings that places hydrogens of one ring above the π-cloud of a neighbor but at a relatively large distance.

The observed aromatic ¹H chemical shifts of compound 2 in FIG. 12(c) vary significantly, ranging from 5.7 to 6.9 ppm, down from 7.2 to 7.8 ppm in solution. In particular, the low 5.7 and 6.3 ppm ¹H chemical shifts of the azobenzene phenylene rings, as probed via their well-resolved nonprotonated carbons near 138 and 143 ppm, indicate non-parallel stacking and aromatic CH—π interactions of these phenylene rings. On the other hand, the terminal phenyl rings show only a small π-shift. The NCH₃ and benzylic CH₂ ¹H chemical shifts in compound 2 are moderately but significantly reduced, see FIG. 15(b); for instance, the NC¹H₃ is slightly lower than the C—C¹H₂-C chemical shift; this is compatible with moderate alkyl CH—π interactions of the NCH₃ and benzylic CH₂ groups.

Alkyl-phenyl proximity in compound 2. ¹H—¹³C HetCor with a long cross-polarization time of 1.1 ms can probe alkyl-aromatic proximity on a scale of about 0.5 nm. The diacetylene with its known structure featuring an azobenzene double layer and alkyl layers, see inset in FIG. 11(a), provides a valuable reference. Its spectrum in FIG. 16(a) shows no cross peak indicating magnetization transfer from alkyl protons near 2.3 ppm to terminal-phenyl-ring carbons near 153 ppm. The near-absence of the corresponding alkyl-to-phenyl peak in compound 1, see FIG. 16(b), indicates segregation of azobenzene and alkyl layers, consistent with the absence of alkyl CH—π shifts in this crystal.

Very distinctly, compound 2 shows a clear (2 ppm, 153 ppm) alkyl-to-phenyl peak in FIG. 16(c) (while the alkyl-diphenyl peak is much weaker). A second strong cross peak between aromatic sites without strong π shift and alkyl ¹H is observed in a vertical cross section near 131 ppm. It can be attributed to the aromatic (C—H)₃ sites of the terminal phenyl ring interacting with alkyl groups; according to the alkyl π-shifts in FIG. 15(b), these are mainly the NCH₃ and benzylic CH₂.

Synopsis of NMR structural analysis. The NMR analysis indicates that compound 1 has azobenzene bilayers with parallel but possibly laterally shifted aromatic rings. The alkyl segments in their layers are kinked in various ways but of limited-amplitude mobility. Hydrogen bonds link the amides flanking the central phenylene ring.

In compound 2, terminal phenyl rings are in proximity of alkyl groups and have moderate alkyl CH—π interactions with NCH₃ and benzylic CH₂ groups. The azobenzene phenylene and the diphenylene rings are subject to significant CH—π interactions with neighboring rings. This excludes azobenzene bilayers in compound 2, unlike in the diacetylene reference and compound 1. The amides of compound 2 are hydrogen-bonded, but the H-bond arrangement is not clear here. The alkyl segments are in an all-anti conformation but the (CH₂)₃ segment that shows no π-interactions exhibits large-amplitude motions; this may be because this segment is in free volume beyond the terminal phenyl rings of neighboring molecules.

Variable-temperature (VT) NMR. 1.05 mg×3 of E-compound 1 or 0.95 mg×3 of E-compound 2 were dissolved in 0.5 mL×3 acetonitrile-d3 (1% TMS)^{[17]}or 0.5 mL×3 methanol-d4 (1% TMS),^{[18]}respectively. VT-NMR of compound 1 were conducted at −40° C., −15° C., and −5° C. VT-NMR of compound 2 were conducted at −5° C., 10° C., and 25° C.

Differential Scanning Calorimetry results are shown in FIG. 18.

Powder X-ray diffraction results are shown in FIG. 19.

UV-vis absorption spectroscopy results are shown in FIG. 20.

Solvent screening and results. To determine solubility, 0.5 mg of compounds were added to 1 mL of common solvents (1-MeOH, 2-acetonitrile (MeCN), 3-acetone, 4-THF, 5-hexane, 6-EtOAc, 7-DMF, 8-DMSO, and 9-toluene, respectively). The samples containing both the compounds and solvent were then organized based on whether suspensions or a clear solution was observed upon addition of the compound. The samples with suspensions were then stirred overnight under 340 nm irradiation. Following this irradiation, a clear solution was observed. These samples were then stirred for 4 hours under 430 nm to trigger the precipitation of the compounds. The solvent that did not precipitate the compounds within 4 hours were deemed unfit for the goal of catalyst recovery. The solvents that were able to reversibly dissolve and precipitate the compounds were then chosen as possible candidates for the recoverable catalyst system and used for catalysis testing.

Quantification of solubility. Excess E-compounds 1-4 were added to 0.5 mL of select solvents. The mixtures were sonicated for 5 min. The obtained suspensions were filtered with a PTFE filter to remove the excess solids. The solvents in clear filtrates were removed using a rotary evaporator and the solutes were dried under vacuum overnight. The solubilities of Z-compounds 1-4 were characterized by activating excess compounds in the solvents upon 340 nm irradiation overnight, then following the same procedure as E isomers. Results are shown in Table 1. In Table 1,

r=[Z]ₘₐₓ/[E]ₘₐₓ

wherein [Z]ₘₐₓ and [E]ₘₐₓ are the maximum concentration of the Z-isomer and the E-isomer, respectively.

TABLE 1

| | | Compound | | | | | | | | | | | | | | | | | |
|---|---|---|---|---|---|---|---|---|---|---|---|---|---|---|---|---|---|---|---|
| | | 1 | | | | 2 | | | | 3 | | | | 4 | | | | | |
| Solvent | | MeCN | | Toluene | | MeOH | | EtOAc | | MeOH | | Acetone | | MeCN | | EtOAc | | Toluene | |
| Isomer (% E) | | 100 | 12 | 100 | 17 | 100 | 16 | 100 | 14 | 100 | 27 | 100 | 14 | 100 | 9 | 100 | 23 | 100 | 55 |
| Solubility | (g/L) | 2.1 | 22 | 5.4 | 15 | 1.9 | 17 | 2.4 | 14 | 3.9 | 16 | 3.8 | 3.3 | 33 | 2.6 | 24 | 2.6 | 36 | |
| | (mM) | 2.9 | 30 | 7.5 | 21 | 2.4 | 21 | 3.0 | 18 | 5.0 | 20 | 4.9 | 4.3 | 43 | 3.3 | 31 | 3.3 | 46 | |

| | | | | | | | | | |
|---|---|---|---|---|---|---|---|---|---|
| r | 10.3 | 2.8 | 8.8 | 6.0 | 4.0 | 5.7 | 10.0 | 9.4 | 13.9 |

Light-induced precipitation of catalysts. To determine light-induced precipitation of catalysts, 44 mg of compound 1 and 34 mg of compound 2 were added to 2 mL of acetonitrile and methanol, respectively. The suspensions were irradiated at 340 nm overnight to obtain clear Z solutions. Then, the solutions were irradiated at 430 nm and monitored over 10 min. Results are shown in FIG. 25.

Thermal reversion kinetics measurements. The thermal half-life of compounds 1 and 2 were determined by following a reported procedure (G. D. Han, et all., J. Mater. Chem. A 2016, 4, 16157-16165). A solution of compounds 1 or 2 (˜1.7×10⁻⁵ M) were prepared in MeCN and MeOH, respectively, then irradiated at 340 nm to obtain a Z-rich solution at photostationary state (PSS), which was then heated at three different elevated temperatures (compound 1: 333 K, 338 K, 343 K; compound 2: 318 K, 323 K, 328 K) in dark. The spectra were recorded over time to monitor the thermal isomerization from Z-to-E. Results are shown in FIG. 26. Table 2 shows a summary of thermal isomerization results (ΔH‡, ΔS‡, ΔG‡, and τ_{1/2}) of Z isomers of compounds 1 and 2 at 298 K.

TABLE 2
Summary of thermal isomerization.

| Compound | 1 | 2 |
|---|---|---|
| ΔH^{‡} (kJ mol⁻¹) | 100.3 | 75.5 |
| ΔS‡ (J mol⁻¹K⁻¹) | −23.8 | −99.7 |
| ΔG^{‡} (kJ mol⁻¹) | 107.4 | 105.2 |
| τ_{1/2} (days) | 8.3 | 3.5 |

### Michael Addition Catalysis.

Kinetic tests of Michael addition reaction.

For compound 1: To a solution of trans-β-nitrostyrene (11.1 mg, 7.5 μmol) and acetylacetone (7.6 mg, 7.5 μmol) in MeCN (0.25 mL) was added either insoluble E isomer or pre-activated Z isomer of compound 1 (5.4 mg, 0.75 μmol). Three identical samples were prepared. 0.2 mL of the solution was taken to check the product yield via ¹H NMR at each time point.

For compound 2: To a solution of trans-β-nitrostyrene (7.9 mg, 5.3 μmol) and acetylacetone (5.3 mg, 5.3 μmol) in MeOH (0.25 mL) was added either insoluble E isomer or pre-activated Z isomer of compound 2 (4.2 mg, 0.53 μmol). Three identical samples were prepared. 0.2 mL of the solution was taken to check the product yield via ¹H NMR at each time point.

The rate constants were calculated based on previous report (M. Sauerland, R. Mertes, C. Morozzi, A. L. Eggler, L. F. Gamon, M. J. Davies, Free Radic. Biol. Med. 2021, 169, 1-11. Results are shown in FIG. 27 and FIG. 28. Table 3 shows absorption coefficient of reactants, product of Michael addition reaction, and catalysts in MeCN and MeOH, respectively.

TABLE 3

| MeCN | Acetylacetone | Nitrostyrene | Product | 1-E | 1-Z |
|---|---|---|---|---|---|
| Conc. (M) | 5.0E−05 | 6.0E−03 | 1.0E−04 | 3.0E−05 | 3.0E−05 |
| ε_{340 nm} | 2.4 | 11327.3 | 59.9 | 24004.3 | 6780.1 |
| (M⁻¹ cm⁻¹) | | | | | |
| ε_{430 nm} | — | — | — | 2336.5 | 2857.4 |
| (M⁻¹ cm⁻¹) | | | | | |
| MeOH | Acetylacetone | Nitrostyrene | Product | 2-E | 2-Z |
| Conc. (M) | 5.0E−05 | 6.0E−03 | 1.0E−04 | 2.0E−05 | 2.0E−05 |
| ε_{340 nm} | 31.1 | 15390.7 | 48.5 | 25623.3 | 2937.1 |
| (M⁻¹ cm⁻¹) | | | | | |
| ε_{430 nm} | — | — | — | 2375.3 | 3632.2 |
| (M⁻¹ cm⁻¹) | | | | | |

NMR analysis of intermolecular interactions between catalysts and Michael addition reactants or product. Three samples of compound 1-E (1.05 mg in 0.5 mL of acetonitrile-d₃, 1% TMS) ^{[17]}and compound 2-E (0.95 mg in 0.5 mL of methanol-d4, 1% TMS) were prepared for each compound. Ten equivalents of each reactant or product were added to each solution. ¹H NMR analysis was conducted to characterize any non-covalent interactions. compound 1 samples were monitored at −5° C., and compound 2 samples at 25° C. Results are shown in FIGS. 29A-29B.

Results of photostability measurements are shown in FIG. 30.

Cycling tests of Michael addition reaction. A four-times larger scale of the above kinetic testing sample was prepared with pre-activated Z isomers of compound 1 or 2 and stirred at room temperature. After that, the reaction mixture was irradiated at 430 nm for 2 hours. The precipitated catalyst was collected by PTFE membrane filtration, further dried under vacuum, and its mass was measured. ¹H NMR was conducted to check the purity of compound 1 or 2 in dry precipitation. The precipitate was used without purification and an equivalent amount of solvent was added based on the mass of the recycled catalyst in the precipitate and irradiated with 340 nm until the solid was fully dissolved. Then, fresh reactants of the same equivalence were added to the solution for the next cycle of reaction.

### Henry Reaction Catalysis.

Kinetic tests of Henry reaction. For compound 1, a solution of 4-nitrobenzaldehyde (11.3 mg, 7.5 μmol) and nitroethane (6.2 mg, 8.2 μmol) in MeCN (0.25 mL) was added to insoluble E isomer or pre-activated Z isomer of compound 1 (5.4 mg, 0.75 μmol) and stirred at room temperature Three same samples were prepared. The same procedure of the kinetic test of Michael addition reaction was followed.

For compound 2, a solution of 4-nitrobenzaldehyde (8.0 mg, 5.3 μmol) and nitroethane (4.4 mg, 5.9 μmol) in MeOH (0.25 mL) was added insoluble E isomer or pre-activated Z isomer of compound 2 (4.2 mg, 0.53 μmol) and stirred at room temperature. Three same samples were prepared, following the same procedure of the kinetic test of Michael addition reaction. The rate constants were calculated based on previous report (D. Kühbeck et al., Beilstein J. Org. Chem. 2013, 9, 1111-1118). Results are shown in FIG. 32 and FIG. 33. Table 4 shows the absorption coefficient of reactants, product of Henry reaction, and catalysts in MeCN and MeOH, respectively.

TABLE 4

| | | Nitro- | | | |
|---|---|---|---|---|---|
| MeCN | Nitroethane | benzaldehyde | Product | 1-E | 1-Z |
| Conc. (M) | 1.0E−03 | 1.0E−03 | 1.0E−03 | 3.0E−05 | 3.0E−05 |
| ε_{340 nm} | 0.3 | 323.3 | 326.5 | 24004.3 | 6780.1 |
| (M⁻¹ cm⁻¹) | | | | | |
| ε_{430 nm} | — | — | — | 2336.5 | 2857.4 |
| (M⁻¹ cm⁻¹) | | | | | |
| | | Nitro- | | | |
| MeOH | Nitroethane | benzaldehyde | Product | 2-E | 2-Z |
| Conc. (M) | 1.0E−03 | 1.0E−03 | 1.0E−03 | 2.0E−05 | 2.0E−05 |
| ε_{340 nm} | 1.3 | 312.5 | 334.6 | 25623.3 | 2937.1 |
| (M⁻¹ cm⁻¹) | | | | | |
| ε_{430 nm} | — | — | — | 2375.3 | 3632.2 |
| (M⁻¹ cm⁻¹) | | | | | |

NMR analysis of intermolecular interactions between catalysts and Henry reaction reactants or product. The procedures used in the Michael reaction were used similarly for Henry reaction. Results are shown in FIGS. 34(a)-34(b).

Cycling tests of Henry reaction. A four-times larger scale of the above kinetic testing sample was prepared with pre-activated Z isomers of compounds 1 or 2 and stirred at room temperature. The same procedure of cycling test for Michael addition reaction as described above was followed for the Henry reaction. Results are shown in FIG. 35A and FIG. 35B.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

The terms “a” and “an” and “the” do not denote a limitation of quantity, but rather denote the presence of at least one of the referenced items. For example, the term “the compound of Formula (I)” means one compound or more than one compound of Formula (I). The term “or” means “and/or” unless clearly indicated otherwise by context. Reference throughout the specification to “an aspect”, means that a particular element (e.g., feature, structure, step, or characteristic) described in connection with the aspect is included in at least one aspect described herein, and may or may not be present in other aspects. In addition, it is to be understood that the described elements may be combined in any suitable manner in the various aspects.

In general, the compositions, methods, and articles can alternatively comprise, consist of, or consist essentially of, any ingredients, steps, or components herein disclosed. The compositions, methods, and articles can additionally, or alternatively, be formulated, conducted, or manufactured so as to be devoid, or substantially free, of any ingredients, steps, or components not necessary to the achievement of the function or objectives of the present claims.

Unless specified to the contrary herein, all test standards are the most recent standard in effect as of the filing date of this application, or, if priority is claimed, the filing date of the earliest priority application in which the test standard appears.

The endpoints of all ranges directed to the same component or property are inclusive of the endpoints, are independently combinable, and include all intermediate points and ranges. For example, ranges of “up to 25 wt %, or 5 to 20 wt %” is inclusive of the endpoints and all intermediate values of the ranges of “5 to 25 wt %,” such as 10 to 23 wt %, etc.

The term “combination” is inclusive of blends, mixtures, alloys, reaction products, and the like. Also, “at least one of” means that the list is inclusive of each element individually, as well as combinations of two or more elements of the list, and combinations of at least one element of the list with like elements not named.

The term “alkyl”, “hydrocarbon”, “saturated hydrocarbon” means an aliphatic hydrocarbon group which may be straight or branched having a recited number of carbon atoms in the chain. Branched means that one or more lower alkyl groups such as methyl, ethyl or propyl are attached to a linear alkyl chain. Exemplary alkyl groups include methyl, ethyl, n- propyl, i-propyl, n-butyl, t-butyl, n-pentyl, and 3-pentyl.

The term “unsaturated hydrocarbon” means alkenyl or alkynyl. The term “alkenyl” means an aliphatic hydrocarbon group containing a carbon-carbon double bond and which may be straight or branched having a recited number of carbon atoms (at least two) in the chain. Branched means that one or more lower alkyl groups such as methyl, ethyl, or propyl are attached to a linear alkenyl chain. Exemplary alkenyl groups include ethenyl, propenyl, n-butenyl, and i-butenyl.

The term “alkoxy” means groups of carbon atoms of a straight, branched, or cyclic configuration and combinations thereof attached to the parent structure through an oxygen. Examples include methoxy, ethoxy, propoxy, isopropoxy, butoxy, cyclopropyloxy, cyclohexyloxy, and the like. Alkoxy also includes methylenedioxy and ethylenedioxy in which each oxygen atom is bonded to the atom, chain, or ring from which the methylenedioxy or ethylenedioxy group is pendant so as to form a ring.

The term “aryl” is inclusive of heteroaryl and means an aromatic monocyclic or multicyclic ring system of 6 to about 14 carbon atoms, preferably of 6 to about 10 carbon atoms. Representative aryl groups include phenyl and naphthyl. The term “heteroaryl” means an aromatic monocyclic ring system of 5 or 6 ring atoms, in which one or more of the atoms in the ring system is/are element(s) other than carbon, for example, nitrogen, oxygen, or sulfur. The prefix aza, oxa, thia, or thio before heteroaryl means that at least a nitrogen, oxygen, or sulfur atom, respectively, is present as a ring atom. A nitrogen atom of a heteroaryl is optionally oxidized to the corresponding N-oxide. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8- tetrahydroimidazo[1,2-a]pyrazinyl, 6,7-dihydro-4H-pyrazolo[5,1-c][1,4]oxazinyl, 2-oxo-2,3- dihydrobenzo[d]oxazolyl, 3,3-dimethyl-2-oxoindolinyl, 2-oxo-2,3-dihydro-1H-pyrrolo[2,3- b]pyridinyl, benzo[c][1,2,5]oxadiazolyl, benzo[c][1,2,5]thiadiazolyl, 3,4-dihydro-2H- benzo[b][1,4]oxazinyl, 5,6,7,8-tetrahydro-[1,2,4]triazolo[4,3-a]pyrazinyl, [1,2,4]triazolo[4,3- a]pyrazinyl, 3-oxo-[1,2,4]triazolo[4,3-a]pyridin-2(3H)-yl, and the like. Preferred heteroaryls include imidazolyl, oxazolyl, isoxazolyl, thiazolyl, and isothiazolyl rings.

The term “halogen” means fluoro, chloro, bromo, or iodo.

The term “substituted” or “substitution” of an atom means that one or more hydrogen on the designated atom is replaced with a selection from the indicated group, provided that the designated atom's normal valency is not exceeded.

“Unsubstituted” atoms bear all of the hydrogen atoms dictated by their valency. When a substituent is keto (i.e., ═O), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture.

Unless defined otherwise, technical and scientific terms used herein have the same meaning as is commonly understood by one of skill in the art to which this invention belongs.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A compound of Formula (I) 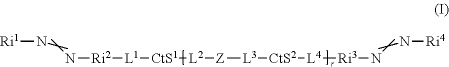
wherein in Formula (I),
Ri¹, Ri², Ri³, and Ri⁴ are each independently an aryl or heteroaryl 5- or 6-membered ring optionally substituted with a C₁₋₆ alkyl, C₁₋₆ alkoxy, C₁₋₆ alkylthio, halogen, cyano, halogenated C₁₋₆ alkyl, halogenated C₁₋₆ alkoxy, or di(C₁₋₆ alkyl)amino;
each L¹, L², L³, and L⁴ is independently a bond or a C₁₋₃₀ linking group optionally including a heteroatom;
CtS¹ and CtS² are each independently catalytic moieties;
Z is a moiety that increases crystallinity of the compound; and
r is an integer from 0 to 4; and
a first geometric isomer of the compound of Formula (I) has a first solubility in the organic solvent, and a second geometric isomer of Formula (I) has a second, different solubility in the organic solvent.

2. The compound of claim 1 of Formula (II) 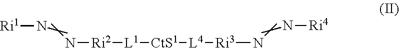
wherein Ri¹, Ri², Ri³, and Ri⁴, L¹ and L⁴, and CtS¹ are the same as in Formula (I).

3. The compound of claim 1 of Formula (III) 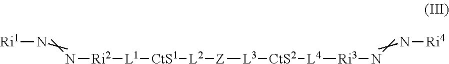
wherein Ri¹, Ri², Ri³, and Ri⁴, L¹, L², L³, and L⁴, CtS¹ and CtS², and Z, and are each as described in claim 1.

4. The compound of claim 1 of Formula (IIIa) 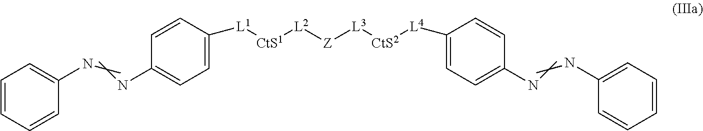
wherein L¹, L², L³, and L⁴, CtS¹ and CtS², and Z, and are each as described in claim 1.

5. The compound of claim 1, wherein at least two of L¹, L², L³, and L⁴ are not a single bond and are a C₁₋₃₀ linking group including a heteroatom.

6. The compound of claim 1, wherein at least two of L¹, L², L³, and are each independently a C₁₋₃₀ linking group including —C(O)O—, —C(O)NH—, —NHC(O)NH—, —NHC(S)NH—, —C(O)NHC(O)— or a C₃₋₃₀ heterocycle.

7. The compound of claim 1, wherein Z is phenyl, naphthyl, anthracenyl, benzo[a]anthracenyl, benzo[a]pyrenyl, perylenyl, chrysyl, fluoranthrenyl, phenanthrenyl, benzo[l]fluorenyl, biphenyl, triphenyl,dialkyne. trialkyne, or a combination thereof.

8. The compound of claim 1, wherein the compound is

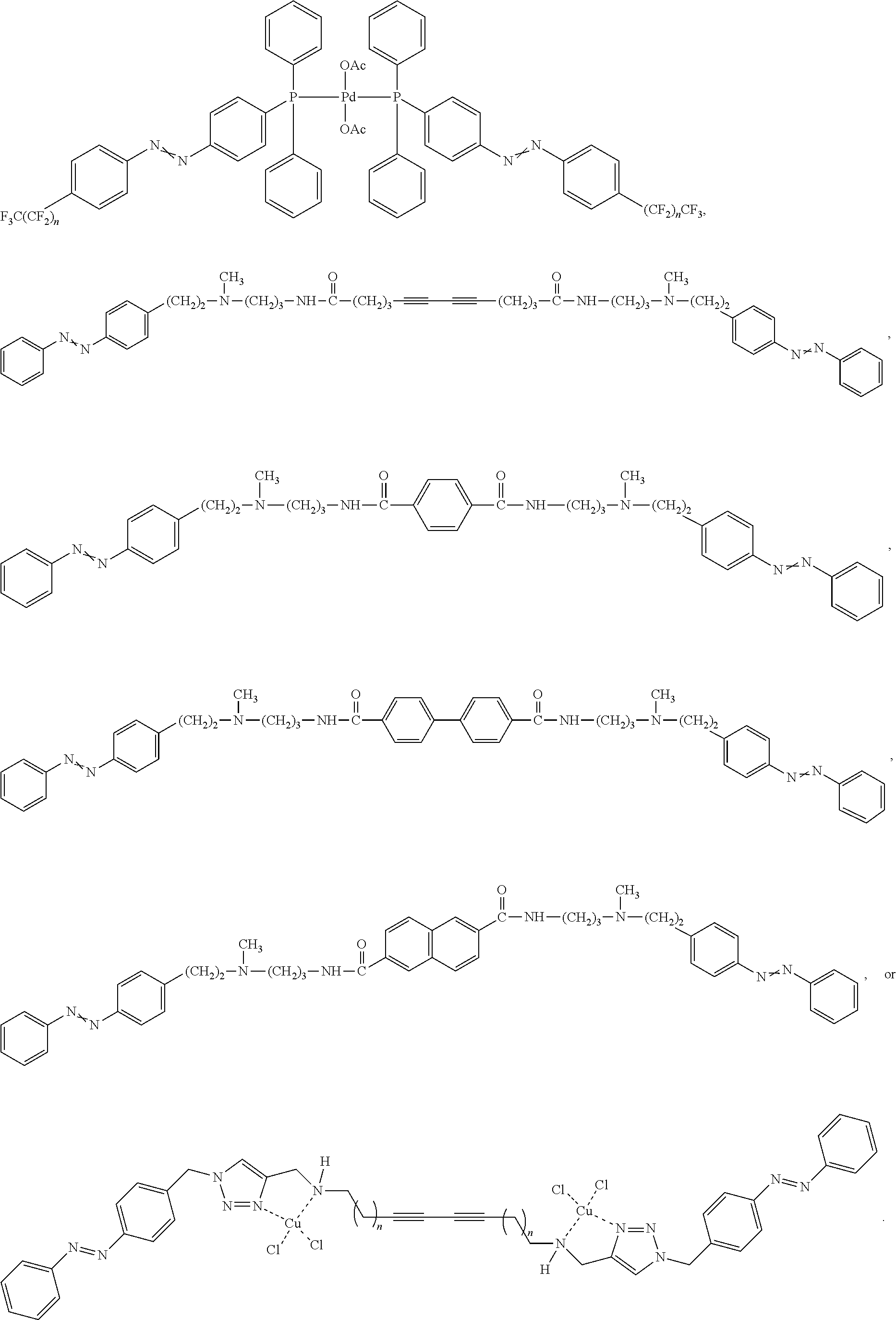

9. A composition comprising the compound of Formula (I) of claim 1, wherein the compound of Formula (I) is predominantly in the form of a Z-isomer or an E-isomer thereof.

10. A method of catalyzing a reaction, comprising:
providing an organic reaction medium comprising the compound of Formula (I) of claim 1 having a first phase having a first solubility in an organic solvent, and a reactant subject to a catalytic transformation by the compound of Formula (I);
irradiating the organic reaction medium with a first wavelength to effect a phase change of the compound of Formula (I) to a second phase having a second, higher solubility in the organic solvent; and
catalyzing the transformation of the reactant to a product.

11. The method of claim 10, further comprising irradiating the second phase of the compound of Formula (I) with light having a second wavelength to effect a phase change from the second phase to the first phase.

12. The method of claim 11, comprising at least two cycles of the providing, irradiating with the first wavelength, catalyzing, and irradiating with the second wavelength.

13. The method of claim 11, further comprising separating the first phase of the compound of Formula (I) from the organic reaction medium to provide a recovered compound of Formula (I) having the first phase.

14. The method of claim 13, comprising at least two cycles of the providing, irradiating with the first wavelength, catalyzing, irradiating with the second wavelength, and separating.

15. The method of claim 11, wherein the light of the first or second wavelength is in the ultraviolet or visible spectrum.

16. A method of catalyzing a reaction, the method comprising:
providing an organic reaction medium comprising
the compound of Formula (I) of claim 1 having a first phase having a first solubility in an organic solvent and a second phase having a higher solubility in an organic solvent than a first phase, wherein the compound of Formula (I) is provided in the form of the second phase, and
a reactant subject to a catalytic transformation by the compound of Formula (I); and
catalyzing the transformation of the reactant to a product by the compound of Formula (I).

17. The method of claim 15, further comprising
after the catalzying, irradiating the compound of Formula (I) in the form of the second phase with light of a second wavelength to effect a phase change of the compound of Formula (I) to the first phase having a lower solubility in the organic solvent;
adding additional reactant to the compound of Formula (I) in the form of the first phase having a lower solubility in the organic solvent; and
before or after adding the reactant, irradiating the compound of Formula (I) in the form of the first phase with light having a first wavelength to convert the compound of Formula (I) to the second phase having a higher solubility in an organic solvent.

18. The method of claim 17, comprising at least two cycles of the providing, catalyzing, irradiating with the second wavelength, adding additional reactant, and irradiating with the first wavelength.

19. The method of claim 17, further comprising
separating the product from the reaction medium including the compound of Formula (I) in the form of the first phase having a lower solubility before adding additional reactant; and
performing at least two cycles of the providing, catalyzing, irradiating with the second wavelength, separating the product, adding additional reactant, and irradiating with the first wavelength.

20. The method of claim 16, wherein the light of the first or second wavelength is in the ultraviolet or visible spectrum.
